(19) United States
(12) Patent Application Publication
(10) Pub. No.: US 2004/0077977 A1
(43) Pub. Date: Apr. 22, 2004

(54) System and method for face and body treatment

(75) Inventors: Sima Ella, Tel Aviv (IL); Haim Rave, Petach Tikva (IL)

Correspondence Address: G.E. EHRLICH (1995) LTD., c/o ANTHONY CASTORINA, SUITE 207, 2001 JEFFERSON DAVIS HIGHWAY, ARLINGTON, VA, 22202, US

(73) Assignee: Engii (2001) Ltd.

(21) Appl. No.: 10/350,222

(22) Filed: Jan. 24, 2003

Related U.S. Application Data
(63) Continuation-in-part of application No. PCT/IL02/00658, filed on Aug. 8, 2002.

(51) Int. Cl. A61H 23/02

(52) U.S. Cl. 601/6; 601/9; 601/15

(57) ABSTRACT

A system and method are provided for self-application of a variety of face and body treatments. Specifically, the system includes a portable control-and-power console and a light-weight portable device having a plurality of spouts, designed for different portions of the body, and comprising different features. The different features are adapted for different modes of treatment, such as suction, macro-massage treatment, lower-chin firming treatment, breast firming treatment, warming light treatment, UV light treatment, LLLT, ultrasound treatment, pulsating magnetic field treatment, constant magnetic field treatment, electrostimulation treatment, cooling-diode treatment, warming-diode treatment, cooling-warming-diode treatment, mechanical epilation, and photoepilation. Additionally, each spout includes at least two, and preferably three or more features, for applying different modes of treatment, in tandem or in sequence, in accordance with predetermined schedules.

### FIELD OF THE INVENTION

The present invention relates generally to devices for face and body treatments, and in particular, to devices, which employ suction-massage treatment, coupled with at least one additional mode of treatment.

### BACKGROUND OF THE INVENTION

Massage devices, which employ suction, are known. Generally, they include a structural casing, which defines a vacuum chamber with an open lower surface, and suction means. The casing is adapted for gliding along a portion of the body. Utilizing a partial vacuum, they cause a lifting of the skin, to form a skin fold. As the device is moved across the body, the skin fold is rolled over by the casing. Thus the device imitates and automates a conventional massaging method, known as “palpate and roll”, which stimulates blood circulation.

Inner rollers may be arranged within the casing, so as to press against the skin folds. These may be associated with motorized drives, for a pinching-like effect, or may rotate freely with the movement of the device.

Massage devices, which employ suction, are described, for example, in U.S. Pat. No. 6,200,281, to Frajdenrajch, entitled, “Massaging device which is designed to be applied to the skin of a person,” U.S. Pat. No. 6,090,055, to Frajdenrajch, entitled, “Massaging Device,” U.S. Pat. No. 6,017,320 to Bleeker, et al., entitled, “Massaging apparatus having two rollers and a suction chamber,” U.S. Pat. No. 5,897,512 to Zagame, entitled, “Massage appliance for moving over a predetermined zone of the human body,” U.S. Pat. No. 3,841,323 to Dan, entitled “Massage Apparatus,” and U.S. Pat. No. 6,196,982, to Ball, entitled, “Vacuum massager,” whose disclosures are incorporated herein by reference.

Additionally, U.S. Pat. No. 5,961,475, entitled, “Massage apparatus with sucking and mobilising action on skin tissue,” and U.S. Pat. No. 5,885,232, entitled, “Roller massaging apparatus with suction function” both to Guitay, both assigned to LPG Systems of France, and both incorporated herein by reference, describe vaccuum massage systems, generally for spas and health centers.

U.S. Pat. No. 4,748,973, to Cho, entitled, “Pneumatic Massage Device,” whose disclosure is incorporated herein by reference, describes a pneumatic massage device, adapted for self-application, without the assistance of another person.

Both U.S. Pat. No. 3,906,940, to Kawada, entitled “Facial Treatment Device,” and U.S. Pat. No. 5,003,966, to Saka, et al., entitled, “Suction device,” whose disclosures are incorporated herein by reference, describe vacuum and massage devices that are adapted for facial treatment.

Another known form of face and body treatment is light treatment, and more specifically, low laser light treatment (LLLT). LLLT refers to laser light at an output of between 1 and 500 Mw. These values are not strong enough to damage chemical bonds in the tissue, but do cause a photochemical effect. LLLT is also referred to as “cold laser” or “soft laser.”

According to “The Healing Nature of light,” http://www.laserpath.com/lpwhatislllt.htm, 1998-2001 LaserPath Therapeutics, downloaded on Apr. 23, 2002, the physiological effects of LLLT include biostimulation, improved metabolism, increase cell metabolism, improved blood circulation and vasodilatation, an analgesic effect, anti-inflammatory and anti-edematous effects, and stimulation of wound healing.

Similarly, according to “Laser Treatment, http://www.skylarkdevice.com/english/laser_e.htm, 2000-2001 Skylark Inc., and according to LLLT, http://www.medsolution.de/LLLT.htm, both downloaded on Apr. 23, 2002, LLLT promotes the production of collagen and the synthesis of protein, accelerates blood circulation, increases lymphatic flow, excites cell division, improves immunity, revives white corpuscles, stimulates the synthesis of Adenosin Tri Phosphate (ATP), and increases the synthetic energy of nucleic acid. Additionally, LLLT can stimulate the skin to improve its tension and elasticity, reduce wrinkles, remove scars, and slow down skin aging.

LLLT penetrates the body to a depth of between 3-5 cm, depending on its wavelength. Light waves in the near infrared ranges penetrate the deepest of all light waves in the visible spectrum.

The recommended wavelength for biostimulation is in the range between 630 and 1200 nm.

U.S. Pat. No. 4,930,504 to Diamantopoulos, et al. entitled, “Device for biostimulation of tissue and method for treatment of tissue,” whose disclosure is incorporated herein by reference, describes a system and method for stimulation of skin and it underlying tissue by laser light of various wavelengths, preferably of at least three different wavelengths. The radiation sources are arranged within the array such that radiation of at least two different wavelengths passes directly or indirectly through a single point located within the treated tissue. The radiation sources are preferably laser diodes, superluminous diodes or similar light-emitting diodes that, while low-power radiation sources, can provide significant energy densities to a treatment area. A method of treatment of tissue comprising exposing the treated tissue to the above-described device is also disclosed. The device for biostimulation of tissue may be included within a system with a control panel, a power source, variable pulse frequency, variable pulse duration, a timer for timing the period of treatment, a device for measuring the conductivity of the treated tissue, a device for measuring the optical power emitted by the radiation sources and (or) a device for detecting emissions from the radiation sources. When three different wavelengths are used, the first wavelength is selected from the group consisting of 650 nm, 660 nm, 680 nm, 750 nm, 780 nm, 800 nm, 810 nm and 820 nm; the second wavelength is selected from the group consisting of 830 nm, 840 nm, 850 nm, 860 nm, 870 nm, and 880 nm; and the third wavelength is selected from the group consisting of 900 nm, 904 nm, 950 nm, 1100 nm, 1300 nm and 1500 nm.

U.S. Pat. No. 5,445,146, to Bellinger, entitled, “Biological tissue stimulation by low level optical energy,” whose disclosure is incorporated herein by reference, describes a method of irradiating a biological tissue of a living subject with optical energy. The wavelength and power dissipation level are such as to cause the amount of optical energy absorbed and converted to heat in the tissue to be within a range bounded by a minimum absorption rate sufficient to elevate the average temperature of the irradiated tissue to a level above the basal body temperature, but which is less than the absorption rate at which tissue is converted into a collagenous substance. According to this method, a therapeutic, warming effect is produced within the irradiated tissue, but without causing tissue damage by thermal overheating. The method of using a low level reactive laser system from 100 milliwatts to 800 milliwatts in either a pulsed or continuous mode with optical energy produced by a Nd:YAG laser at a fundamental wavelength of 1064 nanometers has been found to reduce pain in soft tissues, reduce inflammation and enhance the healing of tissue by stimulation of microcirculation without subjecting the living tissue to damaging thermal effects. The energy density of the irradiated tissue is limited to the range of from about 1 joule per square centimeter to about 15 joules per square centimeter.

Light treatment at other wavelengths is also known. For example, U.S. Pat. No. 4,558,700, to Mutzhas, entitled, “UV Radiation device for phototreatment of dermatoses, especially psoriasis,” whose disclosure is incorporated herein by reference, describes a UV radiation device for phototreatment of dermatoses, especially psoriasis. The device produces UV radiation at a wavelength range below 300 nm at a first intensity, and radiation at a wavelength range between 300 and 310 nm, at a second intensity, substantially higher than the first intensity. The radiation dose being between 0.7 and 1.0 times the erythema threshold dose. Such a UV radiation device is distinguished by good therapeutic effectiveness and the avoidance of undesirable side effects. The power delivered to the skin using Mutzhas' lamp is described as 150W/², which does not have a significant effect on skin temperature.

Similarly, U.S. Pat. No. 4,354,139, to Konijnendijk, et al., entitled, “Low-pressure mercury vapor discharge lamp,” whose disclosure is incorporated herein by reference, describes a low-pressure mercury vapor discharge lamp for radiation purposes. The lamp has a discharge tube made of glass with selective transmission, the tube being coated on the inside with a luminescent layer. This layer contains a luminescent material, which has the characteristic line emission of gadolinium at 312 nm. The discharge tube is made of glass having an absorption edge located between 260 and 280 nm and the tube has at 312 nm a transmission of at least 80%. It is known that radiation in the wavelength range from 305-320 nm may have a favorable therapeutic effect, for example in the treatment of psoriasis and other skin diseases.

U.S. Pat. No. 5,755,751, to Eckhouse, entitled, “Method and apparatus for therapeutic electromagnetic treatment,” whose disclosure is incorporated herein by reference, describes a therapeutic treatment method includes the steps of providing a pulsed incoherent light output for treatment, directing the pulsed light output to a treatment area, transmitting the pulsed light output through the epidermis at the treatment area, heating the epidermis to a temperature below the temperature at which the epidermis is damaged, and coagulating blood in blood vessels disposed below the epidermis.

According to U.S. Pat. No. 5,755,751, hereinabove, when treating vascular disorder, blood content affects the absorption coefficient of the treatment area. Oxyhemoglobin is the main chromophore, which controls the optical properties of blood and has strong absorption bands in the visible region. More particularly, the strongest absorption peak of oxyhemoglobin occurs at 418 nm and has a band-width of 60 nm. Two additional absorption peaks with lower absorption coefficients occur at 542 and 577 nm. The total band-width of these two peaks is on the order of 100 nm. Additionally, light in the wavelength range of 500 to 600 nm is desirable for the treatment of blood vessel disorders of the skin since it is absorbed by the blood and penetrates through the skin. Longer wavelengths up to 1000 nm are also effective since they can penetrate deeper into the skin, heat the surrounding tissue and, if the pulse-width is long enough, contribute to heating the blood vessel by thermal conductivity. Also, longer wavelengths are effective for treatment of larger diameter vessels because the lower absorption coefficient is compensated for by the longer path of light in the vessel.

Accordingly, a wide band electromagnetic radiation source that covers the near UV and the visible portion of the spectrum would be desirable for treatment of external skin and vascular disorders. The overall range of wavelengths of the light source should be sufficient to optimize treatment for any of a number of applications. Such a therapeutic electromagnetic radiation device should also be capable of providing an optimal wavelength range within the overall range for the specific disorder being treated. The intensity of the light should be sufficient to cause the desired thermal effect by raising the temperature of the treatment area to the required temperature. When using pulsed light, the pulse-width should be variable over a wide enough range so as to achieve the optimal penetration depth for each application. Therefore, it is desirable to provide a light source having a wide range of wavelengths, which can be selected according to the desired skin treatment, with a controlled power output and a controlled pulse-width for a particular skin application.

Another known form of face and body treatment is ultrasound treatment. According to “Ultrasound Treatment,” Skylark Inc., http://www.skylarkdevice.com/english/ultrasonic_e.htm#head, ultrasound refers to acoustic waves over 20000 Hz, and in general, ultrasound devices for medical and beauty treatment range in frequency from 1 to 5 MHz. A safe standard for a continuous waveform ultrasound is about 1 W/cm², for about 15 minutes, for general muscle treatment. For facial treatment, it is about 0.5 W/cm², for about 10 minutes. A safe standard for a pulse waveform is about 240 mW/cm². By comparison, shattering kidney stones requires about 10 W/cm². The transmission of ultrasound in air is very low, and generally, an interface of a special gel needs to be used between the ultrasound transducer and the skin.

Ultrasound causes a massaging or vibrating action, which may be regarded as a micro-massaging action. A consequence of it is a heating action. Massaging with 1 MHz for ten minutes may cause a temperature increase of 0.5-1° C., at a tissue depth of 4-5 cm. The warming effect increases blood circulation and tissue metabolism. Ultrasound also causes cavitation of the interface material, which leads to a cleansing of the skin. Additionally, ultrasound increases skin absorption of external matter.

Skylark Incorporated (12th FL., 34, Sec. 3, Chuang Shan N. Rd., Taipei, 104, Taiwan Tel: 886-2-25979005 Fax: 886-2-25912344), carries several products for ultrasound treatment. For example, Ultrasound Treatment Unit (1 MHz) SD-957 is a lightweight device that operates at 1 MHz. It is used for treating deep textures, 2-4 cm into the skin, for example, in regions of the leg, the hip, the back, and the like. Ultrasound Beauty SD-958, a beauty/skin care device, operating at 3 MHz, is specifically adapted for facial treatment.

U.S. Pat. No. 6,325,769, to Klopotek, entitled, “Method and apparatus for therapeutic treatment of skin,” whose disclosure is incorporated herein by reference, describes a method and apparatus which use ultrasound for a therapeutic treatment. According to Klopotek, controlled application of ultrasound energy into the dermis layer reduces the extent of wrinkles and rejuvenate the skin The ultrasound energy triggers a biological response that causes synthesis of new connective tissue in the dermis through activation of fibroblast cells in the dermis without causing or requiring a significant irritation or damage to the epidermis. One purpose of the present method is to provide a cosmetic improvement in the appearance of the skin, meaning that the treated skin surface will have a smoother, rejuvenated appearance, without the need to induce a significant damage to the epidermis layer of the skin.

U.S. Pat. No. 5,665,053 to Jacobs, entitled “Apparatus for performing endermology with ultrasound.” whose disclosure is incorporated herein by reference, describes an endermology body massager having at least two rollers spaced from each other in a parallel configuration. The rollers rotate in the same direction and are mounted on movable axes. A vacuum source is connected to the chamber that houses the rollers. The vacuum source facilitates the suction of the skin between the rollers and helps bring the rollers closer to each other during operation. The rollers or housing have ultrasound generators that are selectively controlled by the operator. In a first embodiment, the ultrasound generators are located within the rollers. In the second embodiment, the ultrasound generators are disposed in the housing around the rollers. Therefore, a controlled and combined endermology with ultrasound treatment can be achieved.

Another known form of face and body treatment is magnetic treatment. According to “Magnetic and electromagnetic treatment,” By David Ramey, www.hcrc.organism/conntriib/ramey/magnet/html, Health Care Reality Check, downloaded on Nov. 20, 2001, researchers believe that a pulsating magnetic field may stimulate biological processes, pertinent for example, to osteogenesis, healing of chronic wounds, and neuronal regeneration.

U.S. Pat. No. 5,669,868, to Markoll, “Treatment of wrinkled discolored or aging skin with magnetic field treatment,” whose disclosure is incorporated herein by reference, describes a process for treating skin by subjecting it to magnetic treatment by an annular coil energized by pulsed D.C. voltage having a rectangular wave form pulsing at the rate of 1-30 CPS, the coil producing a field of under 20 gauss.

Another mode of treatment is epilation, or hair removal. Various manners of epilation are known. For example, the Epilady is a mechanical hair removal device described in http://www.epiladyusa.com, as well as in U.S. Pat. No. 6,277,129, to Poran, entitled “Dual ended hair remover,” whose disclosure is incorporated herein by reference. The Epiladay has a series of high velocity rotating discs, operating like tweezers, that remove hair by the root. Several designs are available, for example, Discrete, and Discrete Plus, both having two speeds, and generally adapted for women's legs, and Lady Bikini, a sensitive trimmer for intimate areas.

Laser based photoepilation is also known. Optical pulses of an appropriate wavelength, pulse duration, and energy density, impinging upon human skin, will result in significant and enduring hair loss. The accepted theory for this phenomenon is that the penetration of the laser into the skin and its subsequent scattering result in heating of the hair shafts and follicles through selective absorption by melanin. The absorption leads to heating of the follicle and subsequent thermal necrosis.

It has been found that for effective photoepilation, the energy must penetrate approximately 3 mm into the tissue. Prevailing thought indicates that this means the absorption should occur in the melanin. Based on the absorption spectrum of melanin, wavelengths in the neighborhood of 700 nm are thought to be efficacious.

U.S. Pat. No. 6,273,885, to Koop, et al., entitled, “Handheld photoepilation device and method,” whose disclosure is incorporated herein by reference, describes a handheld laser tissue treatment device for hair removal. The device comprises a semiconductor diode or diode array laser which emit energy and a device for surface cooling of tissue such that the energy is directed through the cooling device in contact with tissue. The diode laser operates at wavelengths between about 630 nm and 980 nm, and delivers a predetermined amount of energy in a predetermined period of time. Additionally, it has a predetermined spot size. The device utilizes one or more microlenses or microlens arrays to collimate the diode laser energy. Optionally, the device utilizes a deflecting optic for deflecting the diode laser energy through the cooling device which is in contact with tissue in which the deflecting optic is visually transparent such that the operator can see the tissue treatment area. A cooling device such as a sapphire plate or other active or passive cooling means is used to cool the tissue. A method for treatment of tissue, including hair removal, with a handheld device comprises generating laser energy from a semiconductor diode laser, directing the energy through a cooling device in contact with tissue, and treating tissue with the laser energy.

SpaTouch hair removal system, a description of which may be found in http://198.174.40.227/new/radiancy/radiancy, as well as in U.S. Pat. No. 6,214,034, to Azar, entitled, “Method of selective photothermolysis,” and U.S. Pat. No. 6,187,001, to Azar, et al., entitled, “Apparatus and method for removing hair,” whose disclosure are incorporated herein by reference, works on the principle of selective photothermolysis common to other high-tech laser and light-based devices. However, rather than laser, it utilizes pulsed light (in durations of 35 milliseconds) in a wavelength range favorable to melanin, over a very large spot size, for example, about 22×55 mm.

Each of the aforementioned treatment system has specific advantages. A person wishing to benefit from these would have to attend a treatment clinic offering a wide choice of treatment devices, or acquire many different devices, suffering the associated expense and space requirements.

There is thus a need for a device for self-applied face and body treatment, devoid of the above limitations.

### SUMMARY OF THE INVENTION

According to one aspect of the present invention, there is provided a treatment device, comprising:

a spout, which defines an inner chamber, and which is in communication with a vacuum source, for gliding along a portion of a body and applying suction, macro-massage treatment thereto;

at least one feature, integrated with said spout, for applying at least one mode of treatment, different from macro-massage; and a power source, in power communication with said at least one feature.

According to an additional aspect of the present invention, said vacuum source is a pulsating vacuum source.

According to an alternative aspect of the present invention, said vacuum source is a steady vacuum source.

According to an additional aspect of the present invention, said power source is a power line and said device is detachably connected to said power line.

According to an alternative aspect of the present invention, said power source is a self-contained power source.

According to an additional aspect of the present invention, said self-contained power source is rechargeable.

According to an additional aspect of the present invention, said device is detachably connected to said vacuum source.

According to an additional aspect of the present invention, said vacuum source is a vacuum cleaner.

According to an alternative aspect of the present invention, vacuum source is a dedicated vacuum pump.

According to an additional aspect of the present invention, said device includes a gripping handle.

According to an additional aspect of the present invention, said spout is detachably connected to said gripping handle.

According to an additional aspect of the present invention, said device is adapted for self-application.

According to an additional aspect of the present invention, said device further includes at least one knob, for selectively activating and selectively deactivating said at least one feature, for applying said suction, macro-massage and at least one feature individually, and in tandem.

According to an additional aspect of the present invention, said spout further includes at least one roller.

According to an additional aspect of the present invention, said spout further includes at least two rollers.

According to an additional aspect of the present invention, said at least one feature is at least one light source, for applying light treatment to the portion of the body.

According to an additional aspect of the present invention, said at least one light source is operative for photoepilation.

According to an alternative aspect of the present invention, said light treatment is selected from the group consisting of warming light treatment, UV light treatment, LLLT, and a combination thereof.

According to an additional aspect of the present invention, said at least one light source comprises a plurality of light sources of a same type.

According to an alternative aspect of the present invention, said at least one light source comprises a plurality of light sources of different types.

According to an additional aspect of the present invention, said at least one light source is adapted for heating the portion of the body.

According to an alternative aspect of the present invention, said at least one light source comprises a mercury vapor discharge lamp.

According to an alternative aspect of the present invention, said at least one light source comprises at least one laser light.

According to an additional aspect of the present invention, said at least one laser light comprises at least two laser lights of a same wavelength.

According to an alternative aspect of the present invention, said at least one laser light comprises at least two laser lights of different wavelengths.

According to an additional aspect of the present invention, said at least one laser light is a pulsating laser light.

According to an additional aspect of the present invention, said at least one light source is operative at a wavelength range of 200-350 nm.

According to an alternative aspect of the present invention, said at least one light source is operative at a wavelength range of 350-600 nm.

According to an alternative aspect of the present invention, said at least one light source is operative at a wavelength range of 600-700 nm.

According to an alternative aspect of the present invention, said at least one light source is operative at a wavelength range of 700-900 nm.

According to an alternative aspect of the present invention, said at least one light source is operative at a wavelength range of 900-1100 nm.

According to an alternative aspect of the present invention, said at least one light source is operative at a wavelength range of 1100-1500 nm.

According to an alternative aspect of the present invention, said at least one light source is operative at a wavelength of over 1500 nm.

According to an alternative aspect of the present invention, said at least one feature is at least one an electromagnet, for applying a magnetic field treatment to the portion of the body.

According to an additional aspect of the present invention, said magnetic field treatment is selected from the group consisting of pulsating magnetic field treatment and constant magnetic field treatment.

According to an alternative aspect of the present invention, said at least one feature is at least one electrode pair, for applying a electrostimulation treatment to the portion of the body.

According to an alternative aspect of the present invention, said at least one feature is at least one cooling diode.

According to an additional aspect of the present invention, said at least one cooling diode includes at least two cooling diodes.

According to an additional aspect of the present invention, said at least one cooling diode may be flipped so as to operate as a warming diode, for providing warming-diode treatment.

According to an alternative aspect of the present invention, said device further includes at least one warming diode, wherein said cooling diode and said warming diode operate in cycles for cooling-warming therapy.

According to an alternative aspect of the present invention, said at least one feature is at least one mechanical epilation device, for applying mechanical epilation to the portion of the body.

According to an additional aspect of the present invention, said spout includes an additional feature, for applying an additional mode of treatment, selected from the group consisting of warming light treatment, UV light treatment, LLLT, ultrasound treatment, pulsating magnetic field treatment, constant magnetic field treatment, electrostimulation treatment, cooling-diode treatment, warming-diode treatment, cooling-warming-diode treatment, photoepilation, and mechanical epilation, wherein said treatments may be selectively applied in tandem and selectively applied in sequence, in accordance with predetermined restrictions.

According to an additional aspect of the present invention, said treatments may be applied in accordance with a predetermined schedule.

According to another aspect of the present invention, there is provided a treatment system, comprising:

a treatment device, comprising:

a spout, which defines an inner chamber, and which is in communication with a vacuum source, for gliding along a portion of a body and applying suction, macro-massage treatment thereto;

at least one feature, integrated with said spout, for applying at least one mode of treatment, different from macro-massage; and

a power source, in power communication with said at least one feature; and

a power-and-control console, comprising:

an electric pump, in fluid communication with said device, for providing suction;

a system power source, in power communication with said elecrtric pump; and

at least one console control knob, controlling at least said power communication between said pump and said system power source, for activating and deactivating at least said suction.

According to an additional aspect of the present invention, said system power source is in power communication with said device, for providing power to said power source of said device.

According to an additional aspect of the present invention, said at least one mode of treatment is selected from the group consisting of warming light treatment, UV light treatment, LLLT, pulsating magnetic field treatment, constant magnetic field treatment, electrostimulation treatment, cooling-diode treatment, warming-diode treatment, cooling-warming-diode treatment, photoepilation, and mechanical epilation.

According to an additional aspect of the present invention, said spout includes at least two features, for applying at least two modes of treatment, wherein said treatments may be selectively applied in tandem and selectively applied in sequence, in accordance with predetermined restrictions.

According to an additional aspect of the present invention, said treatments may be applied in accordance with a predetermined schedule.

According to an additional aspect of the present invention, said device is detachably connected to said power-and-control console.

According to an additional aspect of the present invention, said system includes a plurality of devices, of different shapes and different modes of treatment.

According to an additional aspect of the present invention, said system includes a plurality of spouts, of different shapes and different modes of treatment.

According to an additional aspect of the present invention, said device includes at least one roller.

According to an additional aspect of the present invention, said device includes at least two rollers.

According to an additional aspect of the present invention, said system further includes a dedicated device to system further includes a dedicated device to firm the lower chin.

According to an additional aspect of the present invention, said system further includes a dedicated device to firm a breast.

According to an additional aspect of the present invention, said system further includes a dedicated device formed as a bra, adapted to firm the two breasts simultaneously.

According to an additional aspect of the present invention, said system further includes a dedicated device to treat impotence.

According to an additional aspect of the present invention, said system is portable.

According to an additional aspect of the present invention, said system power source includes a battery.

According to an additional aspect of the present invention, said system includes a controller.

According to an additional aspect of the present invention, said system includes a display panel.

According to an additional aspect of the present invention, said display panel is interactive.

According to an additional aspect of the present invention, said device is adapted for self-application.

According to still another aspect of the present invention, there is provided a method for face and body treatment, comprising:

applying a first mode of treatment, selected from the group consisting of suction, macro-massage treatment, warming light treatment, UV light treatment, LLLT, pulsating magnetic field treatment, constant magnetic field treatment, electrostimulation treatment, cooling-diode treatment, warming-diode treatment, cooling-warming-diode treatment, photoepilation, and mechanical epilation; and

applying a second mode of treatment, different from said first mode, said second mode of treatment being selected from the group consisting of suction, macro-massage treatment, warming light treatment, UV light treatment. LLLT, pulsating magnetic field treatment, constant magnetic field treatment, electrostimulation treatment, cooling-diode treatment, warming-diode treatment, cooling-warming-diode treatment, photoepilation, and mechanical epilation,

wherein said first and second modes of treatment may be selectively applied in tandem, and selectively applied in sequence, in accordance with predetermined restrictions.

According to an additional aspect of the present invention, said first and second modes of treatment are applied in accordance with a predetermined schedule.

According to an additional aspect of the present invention, said modes of treatment are self-applied.

The present invention successfully addresses the shortcomings of the presently known configurations by providing a system and method for self-application of a variety of face and body treatments. Specifically, the system includes a portable control-and-power console and a lightweight device having a plurality of spouts, designed for different portions of the body, and comprising different features. The different features are adapted for different modes of treatment, such as suction, macro-massage treatment, lower-chin firming treatment, breast firming treatment, warming light treatment, UV light treatment, LLLT, ultrasound treatment, pulsating magnetic field treatment, constant magnetic field treatment, electrostimulation treatment, cooling-diode treatment, warming-diode treatment, cooling-warming-diode treatment, mechanical epilation, and photoepilation. Additionally, each spout includes at least two, and preferably three or more features, for applying different modes of treatment, in tandem or in sequence, in accordance with predetermined schedules.

### BRIEF DESCRIPTION OF THE DRAWINGS

The invention is herein described, by way of example only, with reference to the accompanying drawings. With specific reference now to the drawings in detail, it is stressed that the particulars shown are by way of example and for purposes of illustrative discussion of the preferred embodiments of the present invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice.

In the drawings:

FIGS. 1A-1I schematically illustrate a device for face and body treatment, in accordance with a preferred embodiment of the present invention;

FIGS. 2A-2F schematically illustrate spouts for applying suction to the skin, in accordance with preferred embodiments of the present invention;

FIGS. 3A-3B schematically illustrate spouts, comprising at least one light source, for providing light treatment, in accordance with preferred embodiments of the present invention;

FIGS. 4A-4B schematically illustrate spouts, comprising an ultrasound transducer, in accordance with a preferred embodiment of the present invention;

FIGS. 5A-5B schematically illustrate spouts, comprising an electromagnet, in accordance with preferred embodiments of the present invention;

FIGS. 6A-6C schematically illustrate a spout, comprising electrostimulating electrodes, for providing electrostimulation treatment, in accordance with preferred embodiments of the present invention;

FIG. 7 schematically illustrates a spout, comprising cooling diodes, for providing cooling-diode treatment, in accordance with a preferred embodiment of the present invention;

FIG. 8 schematically illustrates a spout, comprising laser diodes and cooling diodes, in accordance with a preferred embodiment of the present invention;

FIGS. 9A and 9C schematically illustrate spouts, designed for light-induced hair removal, in accordance with preferred embodiments of the present invention;

FIGS. 10A-10B schematically illustrate spouts, designed for mechanical hair removal, in accordance with preferred embodiments of the present invention;

FIGS. 11A-11D schematically illustrate a device and a spout, adapted for use with a standard vacuum cleaner;

FIG. 12 schematically illustrates a spout, adapted for treating impotence;

FIGS. 13A-13C schematically illustrate power-and-control consoles, in accordance with preferred embodiments of the present invention;

FIG. 14 schematically illustrates a system for face and body treatment, in accordance with a preferred embodiment of the present invention;

FIG. 15 is arranged as a table, which delineates predetermined restrictions regarding the different features and different modes of treatment, in accordance with the present invention;

FIGS. 16A and 16B are before and after color photos of a first woman that underwent a treatment, in accordance with the present invention; and

FIGS. 17A and 17B are before and after color photos of a second woman that underwent a vacuum treatment, in accordance with the present invention.

### DESCRIPTION OF THE PREFERRED EMBODIMENTS

The present invention is of a system and method for self-application of a variety of face and body treatments. Specifically, the system includes a portable control-and-power console and a lightweight device having a plurality of spouts, designed for different portions of the body, and comprising different features. The different features are adapted for different modes of treatment, such as suction, macro-massage treatment, lower-chin firming treatment, breast firming treatment, warming light treatment, UV light treatment, LLLT, ultrasound treatment, pulsating magnetic field treatment, constant magnetic field treatment, electrostimulation treatment, cooling-diode treatment, warning-diode treatment, cooling-warming-diode treatment, mechanical epilation, and photoepilation. Additionally, each spout includes at least two, and preferably three or more features, for applying different modes of treatment, in tandem or in sequence, in accordance with predetermined schedules.

The principles and operation of a the device and method according to the present invention may be better understood with reference to the drawings and accompanying descriptions.

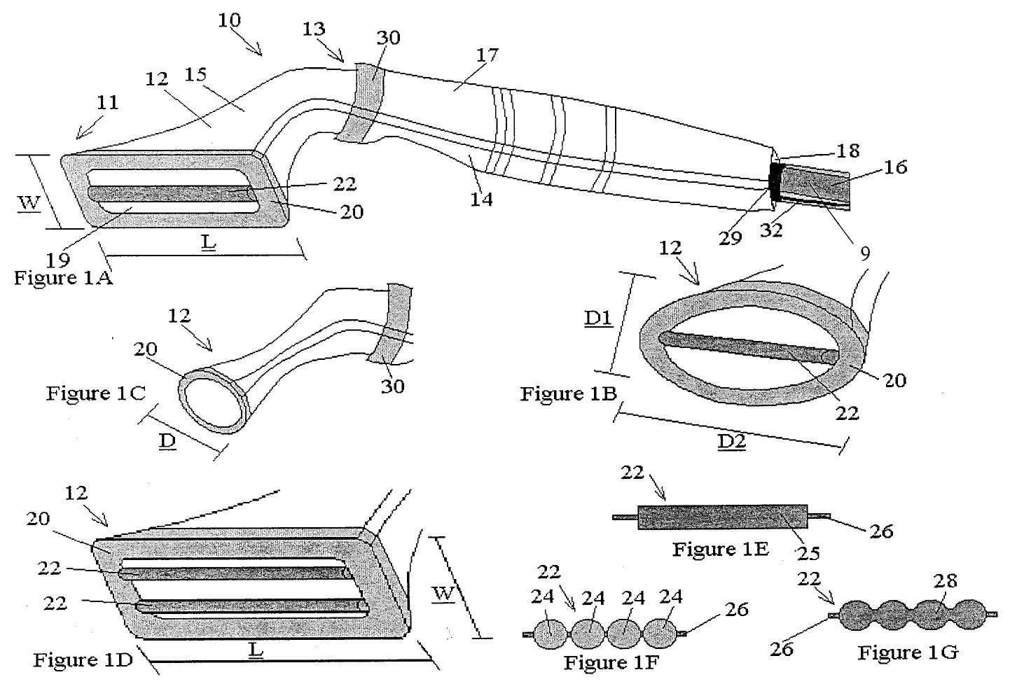

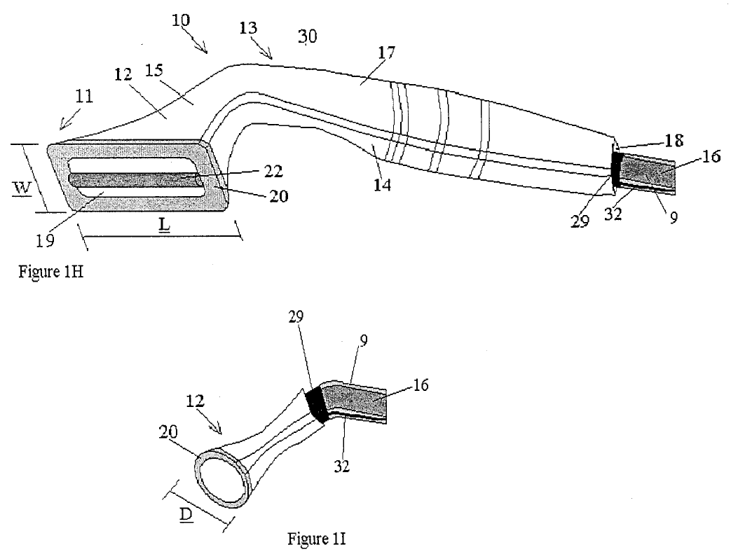

Referring now to the drawings, FIGS. 1A-1I schematically illustrate a device 10 for face and body treatment, in accordance with a preferred embodiment of the present invention. Device 10 is a massage device, which employs suction, imitating the conventional massaging method, “palpate and roll”, and stimulating blood circulation.

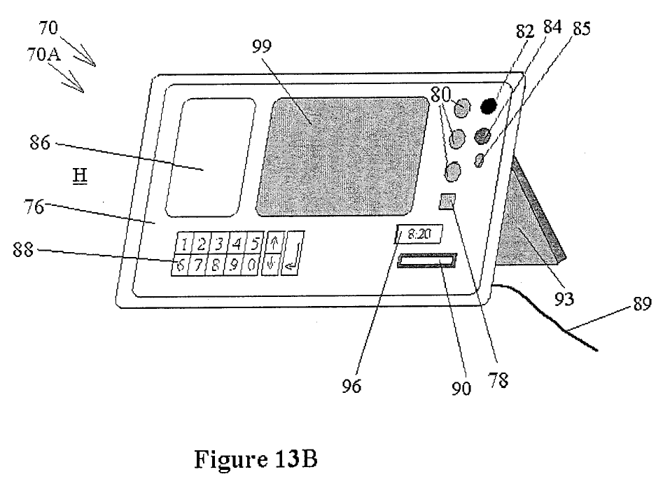

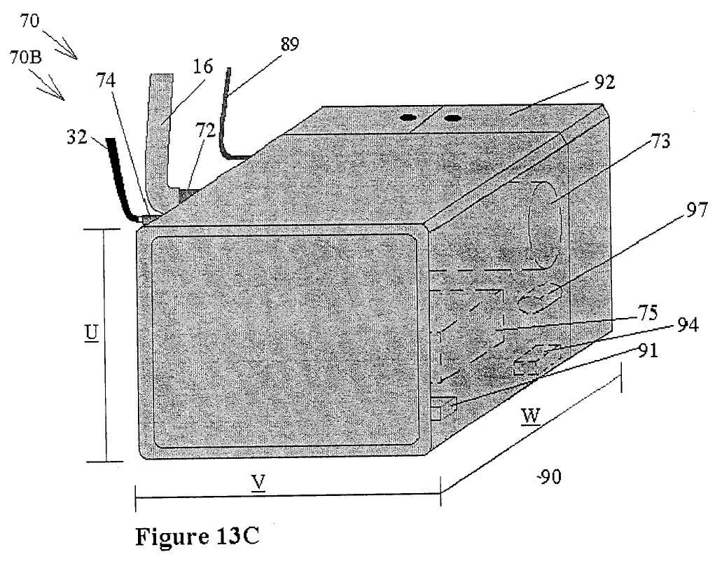

Device 10 includes a spout 12, adapted for gliding along a portion of a body and applying suction thereto. Spout 12 has proximal and distal ends, 11 and 13, with respect to the body, and a casing 15, which defines an inner chamber 19 therein, operative at a pressure below atmospheric pressure, for applying suction to the tissue. Spout 12 is attached to a gripping handle 14, at distal end 13. Gripping handle 14 has a casing 17, which defines a lumen 18, wherein a vacuum hose 16 is arranged, for providing suction to spout 12. Additionally, a power cable 32, described hereinbelow in conjunction with FIGS. 13A-13C, is embedded within casing 15, or within lumen 18. Preferably, vacuum hose 16 and power cable 32 reach device 10 together, enclosed within a sheath 9. Alternatively, they may reach device 10 separately.

Preferably, gripping handle 14 connects with vacuum hose 16 and power cable 32 via a connector 29. Connector 29 may also have a swivel motion, for example of 90 or 120 degrees.

Preferably, device 10 is aesthetically designed and colored. A choice of colors may be provided. Casing 15 of spout 12 and casing 17 of gripping handle 14 may be formed of a rigid plastic, for example, PVC, a combination of wood and plastic, a combination of natural or synthetic rubber and plastic, or the like.

At proximal end 11, spout 12 has a surface 20, which makes contact with the portion of the body under treatment, as device 10 glides along it. Surface 20 may be elliptical, circular, rectangular or of another shape, and is preferably formed as a replaceable pad, preferably of silicon, or another material that is preferably biocompatible, nonallergenic, and soft to the touch. During use, a lubricant, such as a facial cream or a gel may be used between surface 20 and skin 40.

In accordance with a preferred embodiment of the present invention, a plurality of spouts 12 may be provided, of different sizes and shapes, each adapted for a particular portion of the body. For example, for applying suction generally to the face, rectangular spout 12 of FIG. 1A, having a length L substantially of 25 mm, and a width W substantially of 10 mm, with an inner roller of substantially 3 mm in diameter, may be used. Alternatively, an elliptical spout 12 of FIG. 1B, having a minor diameter D1 substantially of 10 mm, and a major diameter D2 substantially of 25 mm, may be used. Alternatively, a 30-mm circular cross section may be used. Yet, for the area under the eyes, circular spout 12, of FIG. 1C, having a diameter of between 4 and 15 mm may be preferred. For applying suction to other portions of the body, for example, the thighs, rectangular spout 12 of FIG. 1D, which is much larger than that of FIG. 1A, substantially of 75 mm in length, and 35 mm in width, may be chosen. It will be appreciated that these choices are individual and different users may select differently. It will be further appreciated that other dimensions may similarly be used.

Spout 12 may be affixed onto gripping handle 14 in a manner similar to that by which different attachments are affixed onto a vacuum cleaner. For example, gripping handle 14 may include a connector 30, operative as a socket, for receiving distal end 13 of spout 12. Preferably connector 30 has a swivel motion, for example of 90 or 120 degrees.

Alternatively, spout 12 may be permanently connected to gripping handle 14.

Additionally, device 10 may include at least one roller 22, arranged at proximal end 11, and protruding proximally from surface 20, for increasing the massaging action to the portion of the body under treatment. As shown in FIG. 1E, roller 22 may be a cylinder 25, arranged on a shaft 26. Alternatively, as shown in FIG. 1F, roller 22 may include balls 24 arranged on shaft 26. Alternatively, as shown in FIG. 1G, roller 22 may have a wavy structure 28, arranged on shaft 26. Alternatively, another roller may be used. Roller 22 may be formed, for example, of rigid plastic, PVC, wood, natural or synthetic rubber, or another suitable material. Alternatively, a bar 22, which does not rotate with the motion of spout 12, may be used.

Preferably, roller 22 may be selectively inserted into spout 12 when a massaging action is desired, and selectively removed from spout 12, for suction without massage. Additionally, spout 12 may be provided with several rollers 22, for example, from amongst those seen in FIGS. 1E-1G, and a user (not shown) may select roller 22 of a desired shape. Furthermore, spout 12 may be adapted to include two or more rollers, as seen in FIG. 1D. Preferably, roller 22 is manually powered, arranged to roll as spout 12 glides along the portion of the body under treatment. Alternatively, roller 22 may be electrically powered.

In accordance with the present invention, the actions of both casing 15 and at least one roller 22 are denoted macro-massage, to distinguish them from electrically assisted forms of treatment that may involve micro-massage, for example, ultrasound.

As seen in FIG. 1H, spout 12 and device 14 may be integrated into a single unit, forming device 10, adapted to fit into connector 29, in a manner similar to that by which different attachments are affixed onto a vacuum cleaner. A plurality of devices 10 may be provided.

Alternatively, gripping handle 14, or device 10, may be permanently connected to vacuum hose 16 and power cable 32.

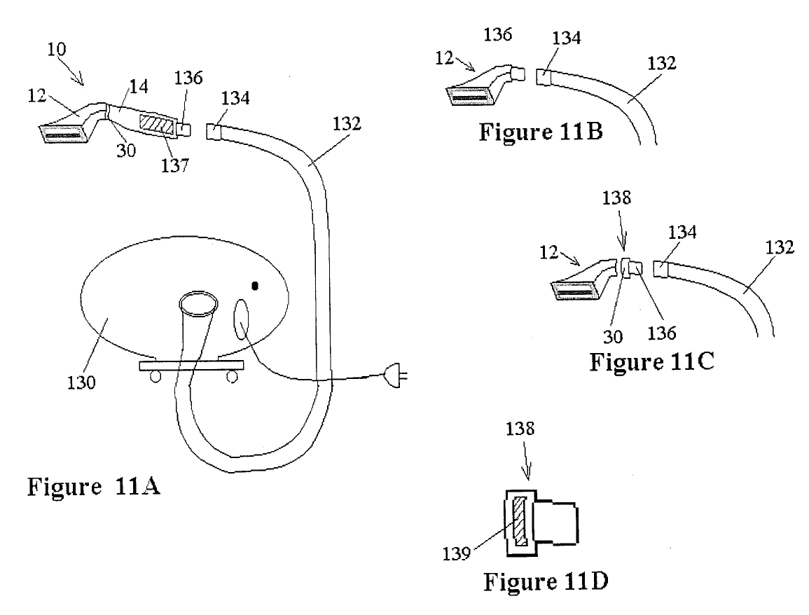

Alternatively as seen in FIG. 11, spout 12 may be directly adapted to fit into connector 29, in a manner similar to that by which different attachments are affixed onto a vacuum cleaner.

Alternatively, spout 12, forming device 10, may be permanently connected to vacuum hose 16 and power cable 32,

may be permanently connected to vacuum hose 16 and power cable 32.

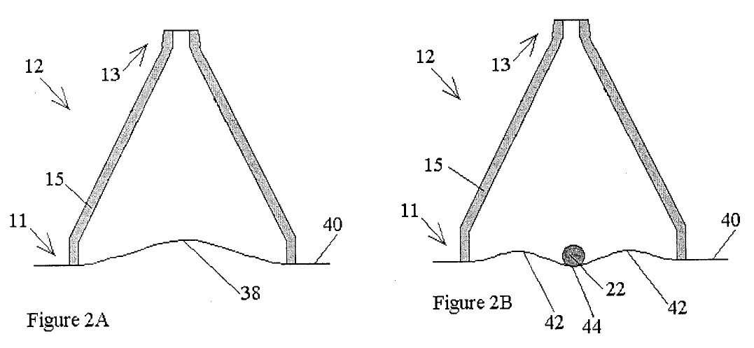

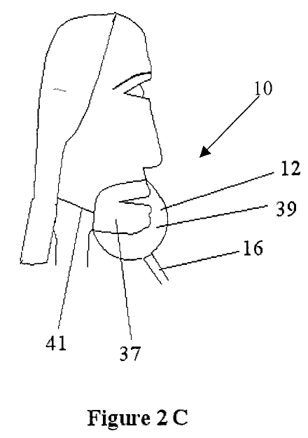

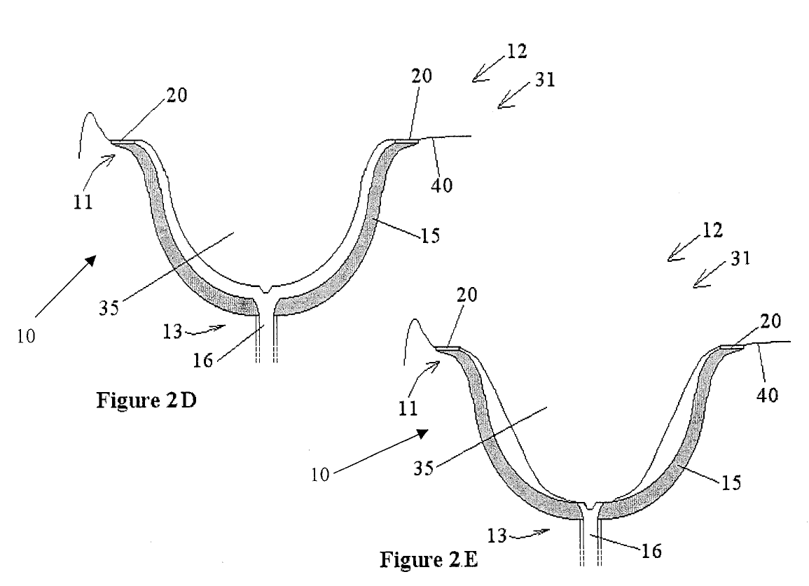

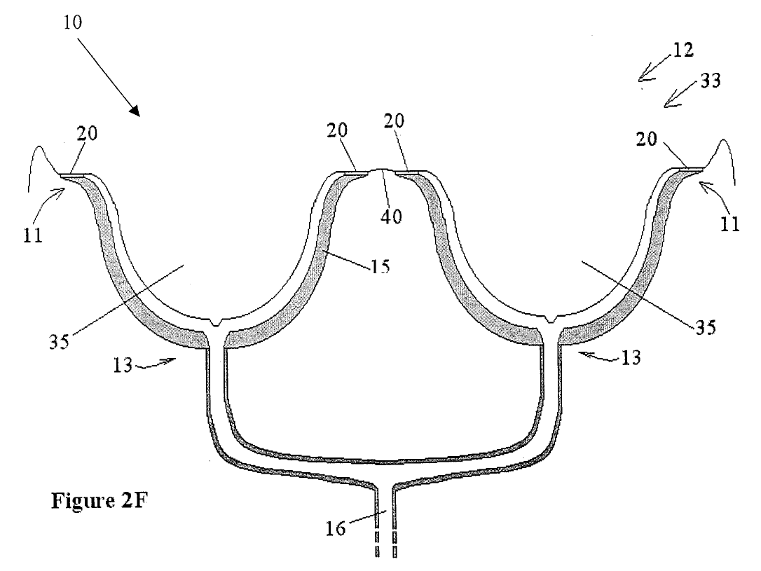

Referring further to the drawings, FIGS. 2A-2B schematically illustrate spouts 12, for applying suction to skin 40, in accordance with preferred embodiments of the present invention.

As seen in FIG. 2A, when suction is applied to skin 40, a skin hump 38 is formed within spout 12. As device 10 is moved across the body, skin hump 38 is rolled over by casing 15, to produce the massaging action.

It will be appreciated that the suction may be steady or pulsating.

As seen in FIG. 2B, when suction is applied, while roller 22 presses down on skin 40, two skin humps 42 are formed, separated by a skin depression 44, that is created by roller 22. Thus both casing 15 and roller 22 participate in the massaging action. It will be appreciated that roller 22 may be motorized or may rotate freely with the movement of spout 12.

It will be appreciated that the vacuum may be a pulsating a steady.

In accordance with the present invention, the actions of both casing 15 and at least one roller 22 will be denoted macro-massage, to distinguish them from electrically assisted forms of treatment that may involve micro-massage, for example, ultrasound.

The range of vacuum may be, for example, between −350 and −500 mbars. Alternatively, it may be, for example, between −250 and −700 mbars. Alternatively, it may be, for example, between −25 and −900 mbars. It will be appreciated that other values may also be used.

The capacity may be, for example, between 200 and 400 liters per minute. Alternatively, it may be, for example, between 100 and 600 liters per minute. Alternatively, it may be, for example, between 50 and 1000 liters per minute. It will be appreciated that other values may also be used.

Clinical tests by LPG, (“Clinical and Skin Tissue Effects of Lift 6®, Dermexpert Laboratory, LPG Systems, France, 2001) show that facial suction treatment, for example by spout 12 of FIG. 2A, tends to firm, and improve loose facial skin. Twenty four women with signs of loose face skin were evaluated clinically. The treatment schedule included 20 sessions during 8 weeks, as follows: three sections a week for the first four-week period and two sections a week for the second four-week period. The clinical evaluations included the following:

1. Cutaneous biopsies of elastic fibers (thin cut) in the papillary dermis showed an increase in oxytalan fibers, after the treatment. The oxytalan fibers were thick, well structured and perpendicular to the dermo-epidermal junction. Additionally the elastic fibers of the papillary dermis, themselves, were better structured.

2. Cutaneous biopsies of elastic fibers (thick cut) showed a thicker elastin network, after the treatment.

3. Cutaneous biopsies (thin cut) in the papillary dermis and superior reticular dermis showed denser collagen networks after the treatment.

4. The number of “crow's foot” wrinkles around the eyes decreased by about 17% and their depth decreased by about 22%, after the treatment.

Additionally, 73% of the women felt there was an improvement in their facial skin quality, after the treatment. For 52% of the women, sufficient improvement was maintained even two weeks after the treatment had ended.

Referring further to the drawings, FIG. 2C schematically illustrates spout 12, for applying suction to a chin and lower face area 37, in accordance with a preferred embodiment of the present invention. Preferably, spout 12 of the present embodiment has a shape 39 which is similar to that of a gas mask. Spout 12 of the present embodiment may be attached with a strap 41.

Clinical tests, by LPG, as described hereinabove, show a lower-chin volume decrease in 54% of the patients, after the four weeks of treatment, and in 70% of the patients after eight weeks of treatment.

Referring further to the drawings, FIGS. 2D-2E schematically illustrate device 10, which includes spout 12, for applying suction to a breast 35, in accordance with a preferred embodiment of the present invention. FIG. 2D illustrates a rest situation and FIG. 2E illustrates breast 35 under suction.

Spout 12 of the present embodiment is shaped as a bra cup 31, having pad 20 in contact with skin 40. Bra cup 31 may be provided in a range of sizes, preferably analogous to bra sizes. Spout 12 of the present embodiment may be attached to the body with strap 41 (FIG. 2C).

Referring further to the drawings, FIG. 2F schematically illustrates device 10, which includes a double spout 12, shaped as bra cups 33, for applying suction simultaneously to two breasts 35, in accordance with another preferred embodiment of the present invention. Pad 20 may form a bridge between bra cups 33. Alternatively, they are maneuvered generally independently. Double spouts 12 of the present embodiment may be attached with strap 41 (FIG. 2C).

Preferably, suction to breasts 35 is applied in pulses, of between 1 and 15 seconds each, and preferably of 2-3 seconds each. The amplitude of the suction pulse may be between −25 and −350 mm Hg. Preferably, the suction pulses have a sharp rise and a relatively slow decay. Alternatively, square or sinusoidal pulses may be used.

Clinical tests by LPG, as described hereinabove, show that suction treatment tends to firm, lift and improve the shape of pendulous breasts. For example, given a treatment schedule of 12 sessions during four weeks (three times a week, for 20 minutes), for 19 Caucasian women between the ages of 26 and 43, 84% of the women reported a lifting effect, that remained even two weeks after the treatment had ended. The lifting effect was expressed by a decrease in the distance between the shoulder and the nipple, from an average of 21.12 cm before the treatment, to 20.41 after two weeks of treatment, 20.55, at the end of the four-week treatment, and 20.68, two weeks after the end of the treatment.

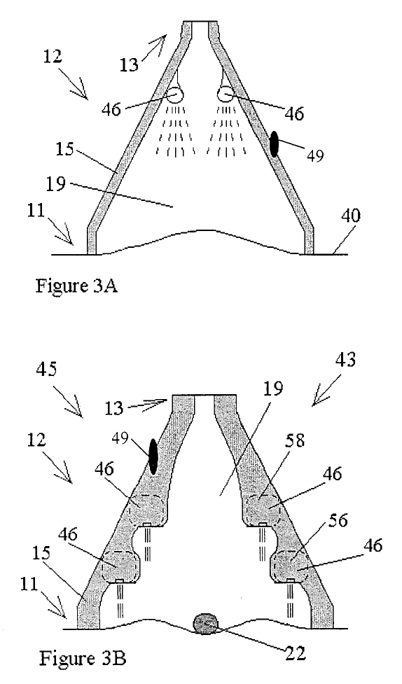

In accordance with the present invention, device 10 further includes at least one feature, integrated with spout 12, for applying at least one mode of treatment, for example, selected from the group consisting of warming light treatment, UV light treatment, Low Level Laser Treatment (LLLT), pulsating magnetic field treatment, constant magnetic field treatment, electrostimulation treatment, cooling-diode treatment, warming-diode treatment, mechanical epilation and photoepilation, as described hereinbelow, in conjunction with FIGS. 3A-15.

Referring further to the drawings, FIGS. 3A-3B schematically illustrate spouts 12, comprising at least one light source 46, for providing light treatment, in accordance with preferred embodiments of the present invention. Light treatment may comprise a warming light treatment, for providing primarily a warming effect to the tissue, UV light treatment, for phototreatment of dermatoses, especially psoriasis, and LLLT. It will be appreciated that a combination of these may be employed, in tandem or in sequence. It will be further appreciated that other forms of light treatment are also possible.

As seen in FIG. 3A, warming light treatment may be provided with at least one light source 46 of one or several incandescent light bulbs, one or several halogen tubes, or one or several infrared light sources, arranged within inner chamber 19 of spout 12. Preferably, a knob 49 may be used to turn off at least one light source 46, so as to apply vacuum only, when desired. It will be appreciated that knob 49 may be located on spout 12 or on gripping handle 14, of device 10.

Alternatively, UV light treatment may be provided, for example by employing light source 46 as a low-pressure mercury vapor discharge lamp, as taught by U.S. Pat. No. 4,354,139, to Konijnendijk, et al., whose disclosure is incorporated herein by reference.

Alternatively, as seen in FIG. 3B, LLLT may be provided by employing at least one light source 46 of one or several laser light sources, for example, laser diodes, preferably imbedded within casing 15. Preferably, knob 49 may be used to turn off at least one light source 46, so as to apply vacuum only, when desired.

According to Skylark Inc., “Laser Treatment, http://www.skylarkdevice.coi/english/laser_e.htm, 2000-2001 and according to LLLT, http://www.medsolution.de/LLLT.htm, both downloaded on Apr. 23, 2002, LLLT promotes the production of collagen and the synthesis of protein, accelerates blood circulation, increases lymphatic flow, excites cell division, improves immunity, revives white corpuscles, stimulates the synthesis of Adenosin Tri Phosphate (ATP), and increases the synthetic energy of nucleic acid. Additionally, LLLT can stimulate the skin to improve its tension and elasticity, reduce wrinkles, remove scars, and slow down skin aging.

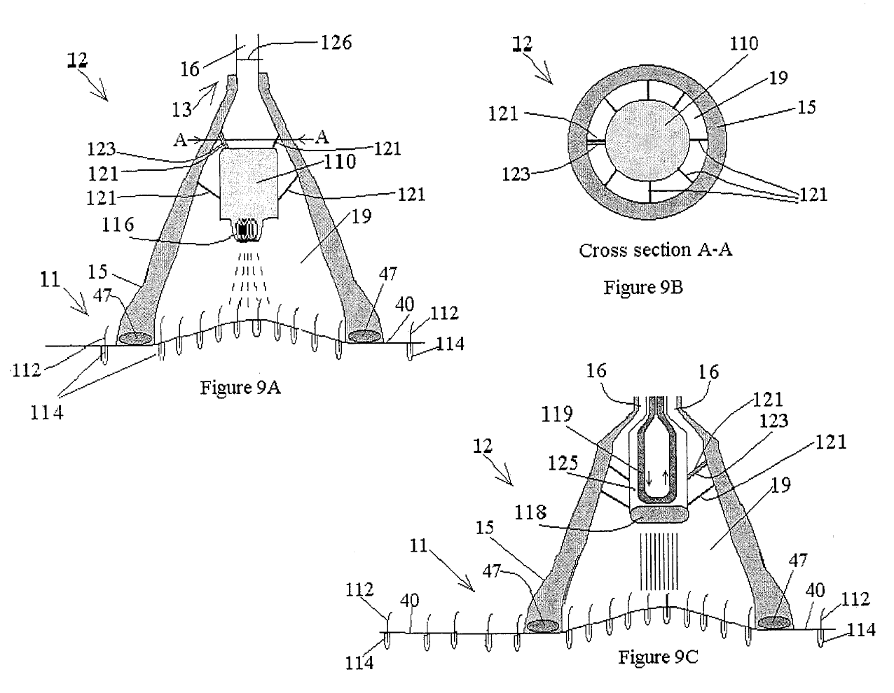

It will be appreciated that a cooling system may be used, for example, as described hereinbelow, in conjunction with FIG. 9B.

As seen in FIG. 3B, the laser light diodes may be arranged in a single tier of diodes, or as two or more tiers of diodes, for example, tiers 56 and 58, wherein each tier of diodes may include between one and four diodes. It will be appreciated that other numbers are also possible. The plurality of Laser light diodes may emit substantially the same wavelength. Alternatively, two or more wavelengths may be used, as taught, for example, by U.S. Pat. No. 4,930,504 to Diamantopoulos, and by U.S. Pat. No. 5,755,751, to Eckhouse, whose disclosures are incorporated herein by reference. For example, tier 56 may operate at a first wavelength, tier 58 may operate at a second wavelength, and additional tiers may be employed, operating at other wavelengths.

The laser light diodes may be pulsed or continuous. When pulsed laser is used, pulsation may take place between a left side 45 and a right side 43, or between tiers of diodes such as tiers 56 and 58. Where more tiers are used, they may be pulsed in sequence. Additionally or alternatively, where two or more wavelengths are employed, they may be pulsed in sequence.

When using laser light source, eye pads (not shown), are worn, for protection. Alternatively, special protective glasses (not shown) may be provided with device 10.

Irradiation by the two or more wavelengths may be simultaneous. Alternatively, it may be sequential. Alternatively, the user may select a predetermined radiation schedule, which may be simultaneous, sequential, or restricted to only one or only two wavelengths, from amongst the wavelengths that are available.

The irradiation wavelength range may be from below 200 to above 1500 nm. Specifically, the range 630-1200 nm may be used for biostimulation. The range 305-320 nm may be used for the treatment of psoriasis and other skin diseases. Additionally, as taught by U.S. Pat. No. 5,755,751, to Eckhouse, other wavelengths for example in the range 400-600, may be employed.

Laser light diodes 46 may be obtained, for example, from Photonic Devices, http://www.photonics.com. Alternatively, they may obtained from Coherent, www.coherent.com, which provide miniature laser diodes, of generally less than 10 mm in diameter and 2-3 mm in height.

It will be appreciated that a cooling system may be used, for example, as described hereinbelow, in conjunction with FIG. 9B.

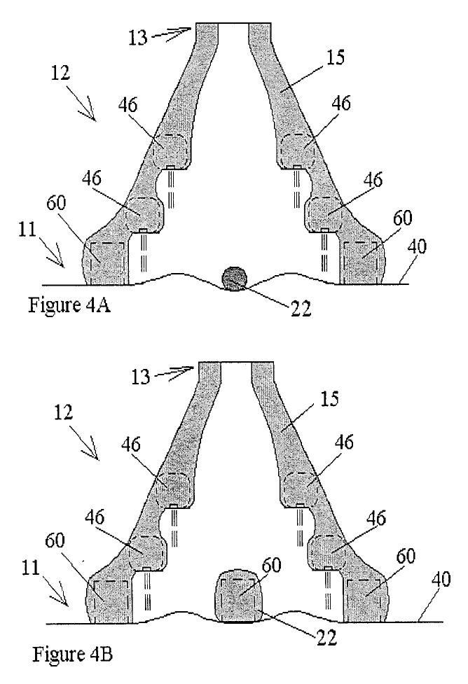

Referring further to the drawings, FIGS. 4A and 4B schematically illustrate spouts 12, comprising an ultrasound transducer 60, in accordance with preferred embodiments of the present invention, for providing ultrasound treatment.

According to “Ultrasound Treatment,” Skylark Inc., http://wvww.skylarkdevice.com/english/ultrasonic_e.htm#head, Ultrasound causes a massaging or vibrating action, which may be regarded as a micro-massaging action. A consequence of it is a heating action. Massaging with 1 MHz for ten minutes may cause a temperature increase of 0.5-1° C., at a tissue depth of 4-5 cm. The waring effect increases blood circulation and tissue metabolism. Ultrasound also causes cavitation of the interface material, which leads to a cleansing of the skin. Additionally, ultrasound increases skin absorption of external matter.

Additionally, according to U.S. Pat. No. 6,325,769, whose disclosure is incorporated herein by reference, controlled application of ultrasound energy into the dermis layer reduces the extent of wrinkles and rejuvenate the skin. The ultrasound energy triggers a biological response that causes a synthesis of new connective tissue in the dermis through activation of fibroblast cells in the dermis without causing or requiring a significant irritation or a damage to the epidermis.

As seen in FIG. 4A, at least one, and preferably several ultrasound transducers 60 may be arranged within casing 15, so as to have direct contact with skin 40. Alternatively or additionally, one or several ultrasound transducers 60 may be arranged within roller 22, or bar 22, so as to have direct contact with skin 40. During use, a lubricant, such as a facial cream or a special gel may be used between surface 20 (FIG. 1A) and skin 40. Alternatively, collagen cream, or another beneficial cream may be used and absorbed by the skin as a result of the ultrasound action.

When using a continuous waveform, the frequency range may be about 1-5 MHz, at a recommended power of about 1 W/cm², for a treatment time of about 15 minutes, for general muscle treatment. The recommended power is about 0.5 W/cm², and the recommended treatment time is about 8-10 minutes, for facial treatment. Alternatively, when using a pulse waveform, the recommended power is about 240 mW/cm², for about 15 minutes, for general muscle treatment, and about 120 mW/cm²W/cm², for about 8-10 minutes, for facial treatment. It will be appreciated that other values may be used.

Ultrasound transducer 60, in the frequency range between 50 kHz and 2 MHz, at sizes as small as 0.2 in. external diameter and 0.36 in. in height, may be obtained, for example, from Digital Wave Corporation, 11234-A East Caley Av., Englewood, Colo. 80111 USA, Tel 303-790-7559, Fax. 303-790-7567, www.digitalwave.com. It will be appreciated that other values may be used.

As seen in FIGS. 4A and 4B, spout 12 may further include at least one light source 46, which may be any of the light sources described hereinabove, in conjunction with FIGS. 3A-3B. Preferably, the user may select either the a light treatment, such as LLLT treatment, or the ultrasound treatment, or both, in tandem or in sequence.

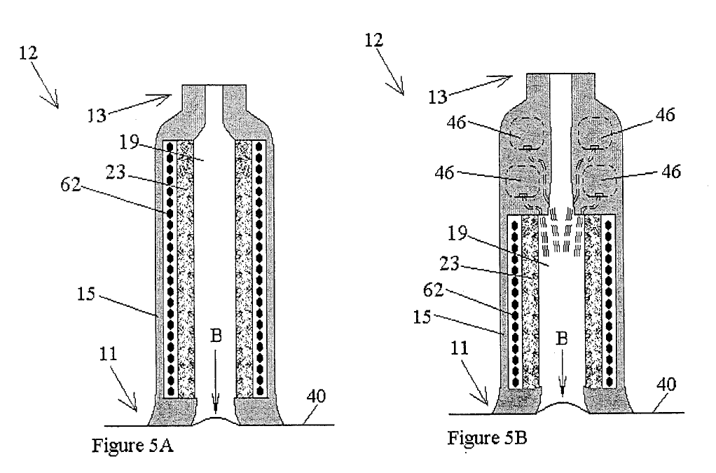

Referring further to the drawings, FIGS. 5A and 5B schematically illustrate spouts 12, comprising an electromagnet 62, for providing pulsating magnetic field treatment, or constant magnetic field treatment, in accordance with preferred embodiments of the present invention.

According to “Magnetic and Electromagnetic Treatment,” By David Ramey, www.hcrc.organism/conntriib/ramey/magnet/html, Health Care Reality Check, downloaded on Nov. 20, 2001, some researchers believe that pulsating magnetic field may stimulate biological processes, pertinent for example, to osteogenesis, healing of chronic wounds, and neuronal regeneration.

Preferably, electromagnet 62 comprises a conducting wire coil 62, arranged within casing 15. Additionally, an inner casing 23, preferably formed of a molded rigid plastic containing iron beads, iron filings, or the like, for example at a ratio of 1 part iron to 3 parts plastic, may be used on the side of conducting wire coil 62 facing inner chamber 19, to increase the magnetic field generated by conducting wire coil 62. Alternatively, another ratio may be used. Alternatively, no iron beads may be used. When current flows in wire coil 62, a magnetic field B is formed. To generate a pulsating magnetic field, a pulsed DC voltage may be used. For example, a pulsed DC voltage having a rectangular wave form, pulsing at the rate of 10-60 cycles per minutes (CPM), the coil producing an average field of between 5 and 25 gauss, may be used It will be appreciated that other values may be used.

Alternatively, a constant DC voltage may be used, to form a constant magnetic field.

In accordance with the present invention, a permanent magnet may be used in place of wire coil 62, to form a constant magnetic field.

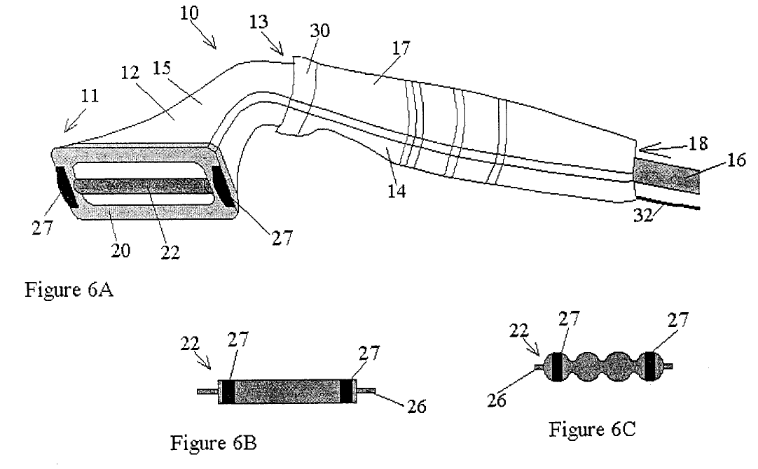

Referring further to the drawings, FIGS. 6A-6C schematically illustrate spout 12, comprising electrostimulating electrodes 27, for providing electrostimulation treatment, in accordance with preferred embodiments of the present invention.

As seen in FIG. 6A electrostimulating electrodes 27 may be arranged on surface 20. Alternatively, as seen in FIGS. 6B and 6C, electrostimulating electrodes 27 may be arranged on roller 22. Electrodes 27 may be formed, for example, of stainless steel, titanium, or titanium alloy or another preferably noncorrosive, biocompatible conducting metal.

Preferably, electrostimulation treatment is performed at between 16 and 64 V, and up to 15 mA. It will be appreciated that other values may be used.

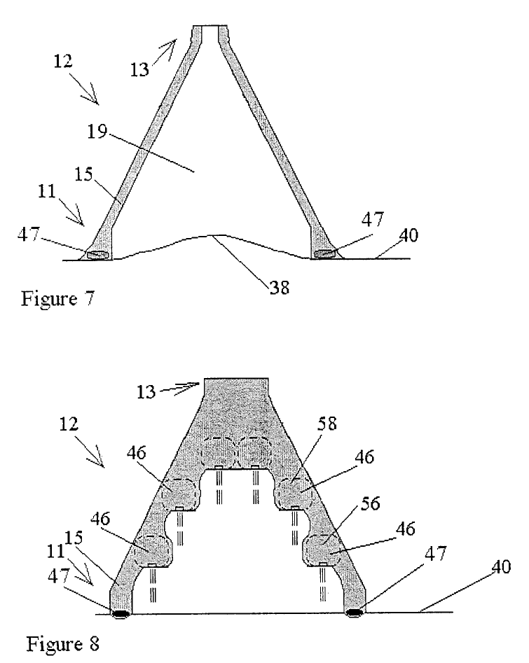

Referring further to the drawings, FIG. 7 schematically illustrates spout 12, comprising cooling diodes 47, arranged on surface 20, for providing cooling-diode treatment, in accordance with a preferred embodiment of the present invention. Cooling-diode treatment may be desired, for example, to close the skin pores, at a conclusion of a treatment. Alternatively, cycles of heating and cooling may be employed, by combining for example, warming-light treatment and cooling-diode treatment.

Additionally, in accordance with an embodiment of the present invention, cooling diodes 47 may be selectively flipped by the user, when desired, so as to operate as warming diodes, for providing a warming effect, or warming-diode treatment.

Preferably, diodes 47 operate at between 7-10° C. colder or warmer than the skin. However, other temperature values are also possible. Preferably, diodes 47 operate at 12 V and up to 7 amp. It will be appreciated that other values may be used.

Referring further to the drawings, FIG. 8 schematically illustrates spout 12, comprising laser diodes 46, embedded within casing 15, and cooling diodes 47, arranged on surface 20, for providing LLLT, in combination with cooling-diode treatment, in accordance with a preferred embodiment of the present invention. It will be noted that spout 12 of FIG. 8 is not adapted for applying the suction, macro-massage of FIGS. 2A-2B, but only other modes of treatment. Similarly, spout 12 may be adapted for light treatment with electrostimulation treatment, or ultrasound treatment with cooling diode treatment, or other combinations, in tandem or in sequence. Spout 12 may be further adapted for a combination of three or more modes of treatment, which may or may not include the suction, macro-massage of FIGS. 2A-2B.

Referring further to the drawings, FIGS. 9A and 9C schematically illustrate spouts 12, designed for light-induced hair removal, or photoepilation, in accordance with a preferred embodiment of the present invention.

As seen in FIGS. 9A and 9B, a photoepilation hair-removal device 110 may be attached to casing 15 of spout 12 by a structure 121, arranged for example, as a grid, so as not to block the suction in inner chamber 19. Additionally, power may be supplied to hair-removal device 110, via a powerline 123, which may be embeded within structure 121.

Photoepilation hair-removal device 110 is adapted to burn hairs 112 and roots 114, on skin 40. Preferably, photoepilation hair-removal device 110 is similar to the SpaTouch, described in http://198.174.40.227/new/radiancy/radiancy, as well as in U.S. Pat. No. 6,214,034, to Azar, entitled, “Method of selective photothermolysis,” and in U.S. Pat. No. 6,187,001, to Azar, et al., entitled, “Apparatus and method for removing hair,” whose disclosures are incorporated herein by reference. Thus, spout 12 of the present embodiment comprises a preferably pulsed light 116, which pulsates for example, in durations of 35 milliseconds, at a wavelength range favorable to melanin, over a very large spot size, for example, about 22×55 mm. It will be appreciated that other values may be used. Pulsed light source 116 may be a quarts light source.

It will be appreciated that although spout 12 seen in FIG. 9B is circular, other shapes, for example, rectangular, or elliptical may be used.

It will be appreciated that cooling diodes 47 may be used against skin 40, to allevaiate the heat, generated by the photoepilation.

In accordance with the present invention, spout 12 applies suction to skin 40, for sucking the burnt hair ashes, through vacuum hose 16. A removable filter 126 may be used for collecting the ashes.

Spout 12 of the present embodiment can treat individuals with most skin and hair types. The procedure works best, however, for people with dark brown or black hair and a fair complexion because of the color contrast and the fact that darker hair has a higher melanin content. Areas of the body that have been exposed to the sun or ultraviolet light in tanning booths should not be treated until one month after exposure.

Since dark colored hair tends to absorb light and burn more effectively than light colored hair, the user may dye hair 112, with a dye that infiltrates roots 114, prior to burning it. The dye may be a hair dye, as known, or liquid carbon, also known as Ferrite.

As seen in FIG. 9C, a laser photoepilation hair-removal device 125 may be attached to casing 15 of spout 12, for example, by structure 121. Additionally, power may be supplied to device 125 via powerline 123, which may be embeded within structure 121.

Laser photoepilation hair-removal device 125 may be basically similar to that of U.S. Pat. No. 6,273,885, to Koop, et al., Handheld photoepilation device and method, whose disclosure is incorporated herein by reference. Spout 12 of the present embodiment may thus inlcude a semiconductor diode 118 or diode array laser 118 which emit energy at wavelengths prefrably between about 630 nm and 980 nm, and delivers a predetermined amount of energy in a predetermined period of time. It will be appreciated that other values may be used.

Photoepilation hair-removal device 125 may include a cooling system 119, for example of water, air, or another fluid. The coolant system may be arranged within vacuum hose 16. Alternatively, spout 12 may include a finned, high heat-capacity material, as a heatsink, for passive cooling. Alternatively, another cooling system may be used, for example, cooling diodes.

It will be appreciated that powerline 123 may be arranged along cooling system 119.

Additionally, as in FIG. 9A, cooling diodes may be used against skin 40, to allevaiate the heat generated by the photoepilation.

In accordance with the present invention, spout 12 applies suction to skin 40, for sucking the burnt hair ashes.

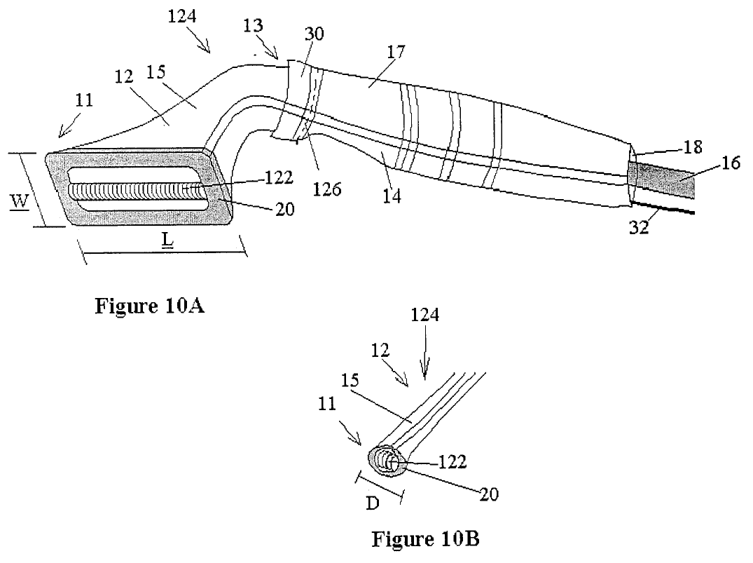

Referring further to the drawings, FIGS. 10A-10B schematically illustrate spouts 12, designed as the Epilady, described in http://www.epiladyusa.com, as well as in U.S. Pat. No. 6,277,129, to Poran, entitled, “Dual ended hair remover,” whose disclosure is incorporated herein by reference, for mechanical hair removal, in accordance with a preferred embodiment of the present invention.

Thus, spout 12 of the present embodiments, operative as mechanical hair-removal device 124, has a series of high velocity rotating discs 122, operating like tweezers, that remove hair by the root. FIG. 10A illustrates a basic design, adapted for example, for women's legs, and FIG. 10B illustrates a miniature design, adapted for example, for facial hair, or as a sensitive trimmer for intimate areas.

In accordance with the present invention, inner chamber 19, around series of high velocity rotating discs 122, applies suction to the tissue, for sucking the hair away, through vacuum hose 16. A removable hair trap 126, or a hair filter 126, may be used for removing the hair.

Mechanical hair-removal device 124 may be attached to casing 15 of spout 12 by a structure 121, for example, as taught hereinabove, in conjunction with FIGS. 9A and 9B. Structure 121 is strong enough to withstand the vibrations of the mechanical epilation. Additionally, power may be supplied to hair-removal device 124, via powerline 123, embeded within structure 121 (9A and 9B).

Referring further to the drawings, FIGS. 11A-11D schematically illustrate device 10 and spout 12, adapted for use with a standard vacuum cleaner 130, in accordance with preferred embodiments of the present invention.

As seen in FIG. 11A, device 10 may include a fitting 136, to enable use with a standard vacuum cleaner hose 132, having a fitting 134. Handle 14 of device 10 may further include a battery 137, for providing power to other features of spout 12, such as ultrasound or LLLT. Battery 137 may be rechargeable.

Alternatively, as seen in FIG. 11B, spout 12 may include fitting 136, to enable use with standard vacuum cleaner hose 132, having fitting 134.

Alternatively, as seen in FIG. 11C, a special adapter 138 may include fittings 136 and 134, to enable use with standard vacuum cleaner hose 132.

As seen in FIG. 11D, special adapter 138 may further include a battery 139, for providing power to other features of spout 12, such as ultrasound or LLLT. Battery 139 may be rechargeable.

Vacuum cleaner 130 may be a canister type vacuum cleaner, a brush type vacuum cleaner, a beater type vacuum cleaner, a hand-held, light, portable vacuum cleaner, such as a dust buster, a central, built-in vacuum cleaner, or any other vacuum cleaner, as known.

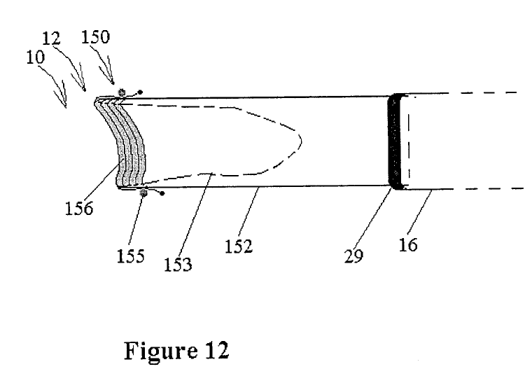

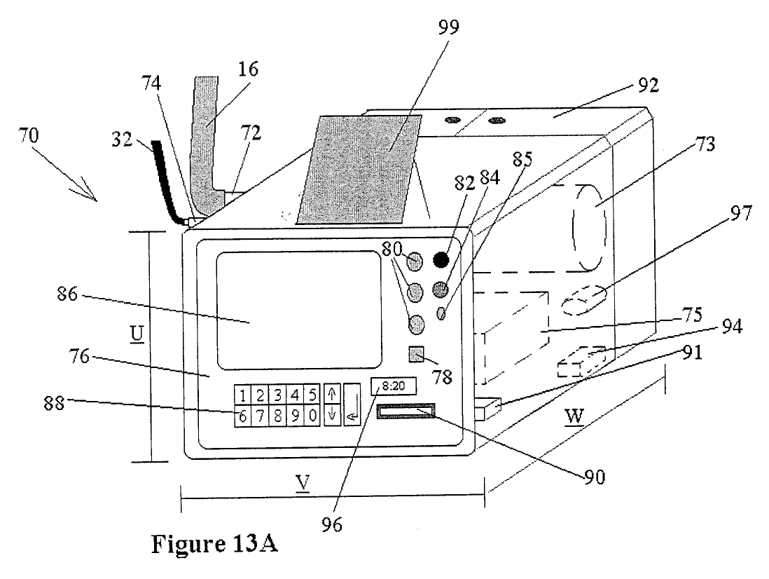

Referring further to the drawings, FIG. 12 schematically illustrates a spout, adapted for treating impotence, in accordance with an embodiment of the present invention.

It is known that the application of vacuum to the penis is operative to bring about an erection. Spout 12 may be designed as an erection implement 150, basically shaped as a soft, flexible hose 152. Additionally, erection implement 150 may further include a mouthpiece 156, adapted to form a seal with the body. Preferably, mouthpiece 156 is soft and flexible, formed for example, as an accordion, so as to adapt to the body shape. Erection implement 150 may be adapted to connect with vacuum hose 16 and power cable 32 via connector 29.

In order to prevent fluids from reaching vacuum hose 16, a condom 153 may be used, inserted into erection implement 150, folded over the edges of mouthpiece 156, and held, for example, with a rubber band 155.

Referring further to the drawings, FIG. 13A schematically illustrates power-and-control consoles 70, in accordance with a preferred embodiment of the present invention. Preferably, power-and-control console 70 is elegantly and attractively designed and colored. A choice of colors may be provided.

Power-and-control console 70 provides vacuum to vacuum hose 16, via a vacuum line 72, in communication with a vacuum source 73, and power to cable 32, via a power line 74, preferably receiving power from a power supply unit 75. Additionally, power-and-control console 70 preferably includes a control panel 76, having an on/off control knob or control button 78 and preferably also several other knobs or buttons 80. Knobs 80 may be used to selectively activate different modes of treatment such as suction, macro-massage treatment, ultrasound treatment, pulsating magnetic field treatment, LLLT and others. Preferably, knobs 80 may also control the intensity and other variables of the treatment. For example, when ultrasound treatment is selected, knobs 80 may be used to select the desired frequency, the desired power range, and the desired duration of treatment. Similarly, when LLLT is selected, knobs 80 may be used to select the desired wavelength or wavelengths, the desired power range, and the desired duration of treatment. Additionally, knobs 80 may be used to select a treatment schedule, such as LLLT with suction massage, or LLLT directly after the suction massage, or another treatment schedule.

Knobs 78 and 80 may also be located directly on gripping handle 14. This allows the user to initially turn device 10 on from power-and-control console 70, but pause or change treatment modes directly from gripping handle 14.

Additionally, control panel 76 may also include a display panel 86, which may display the feature or schedule that was selected. Display panel 86 may be interactive, so the user may touch it to select features or schedules. Alternatively or additionally, a keyboard 88 may be provided, for selecting a desired feature or schedule.

Power-and-control console 70 may further include a controller 91 or a microcomputer 91, which may also include a memory. A read/write storage system 90 such as a diskette drive, a CD drive, or a mini CD drive may be used for storing or reading specific schedules.

Furthermore, power-and-control console 70 may include a clock 96, which is preferably digital, to clock the duration of a treatment.

Additionally, power-and-control console 70 may include a preferably rechargeable battery 94.

Preferably, control panel 76 also includes a light 82, for indicating that the system is on. Preferably a second light 84 may be provided as a warning light, for example, when the treatment time, or requested power level exceeds a recommended value. When this happens, controller 91 may stop the treatment.

Additionally, power-and-control console 70 may include a thermostat. A third light 85 may serve as a warning light, to indicate when power-and-control console 70 overheats. When this happens, controller 91 may stop the treatment.

Furthermore, power-and-control console 70 may include a short-circuit protection 97.

Preferably, a length V of power-and-control console 70 is 36 cm, a width W is 24 cm, and a height U is 13 cm. It will be appreciated that other dimensions are possible.

Power-and-control console 70 may further include a storage box 92 for storing accessories such as different spouts 12 and rollers 22, and a mirror 99, for allowing the user to view a facial treatment. Preferably, mirror 99 may be folded flat.

Referring further to the drawings, FIGS. 13B-13C schematically illustrate power-and-control console 70, in accordance with another preferred embodiment of the present invention. Preferably, power-and-control console 70 is formed of a control-and-display portion 70A and a power-and-vacuum portion 70B. Power-and-vacuum portion 70B may be stored in a cabinet, or under a table, while control-and-display portion 70A, which may further include a support back 93, may be easily accessible—kept over a table, or over chest, or hung on the wall. Control-and-display portion 70A is preferably elegantly and attractively designed and colored. A cable 89 may be used to connect control-and-display portion 70A and power-and-vacuum portion 70B.

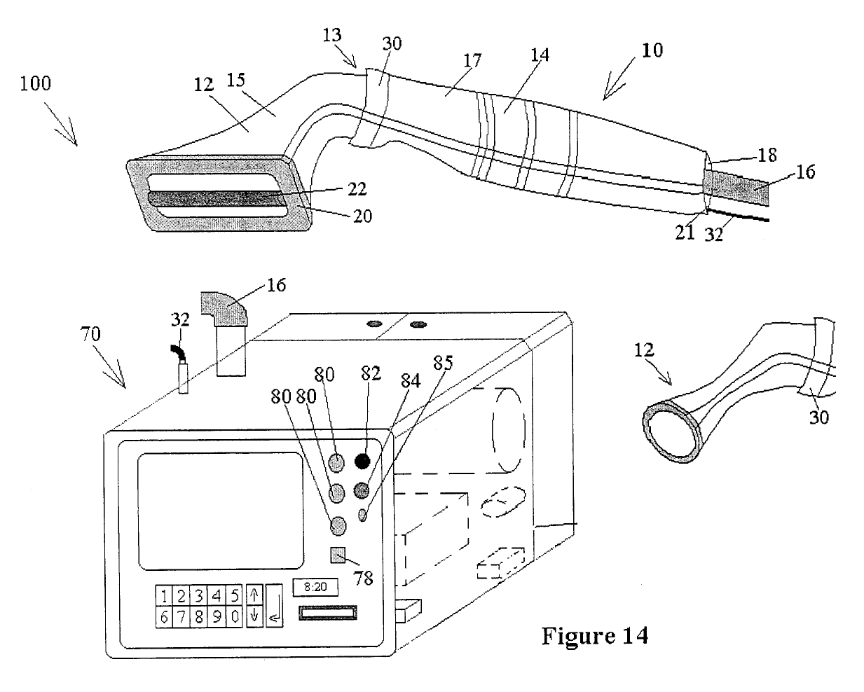

Referring further to the drawings, FIG. 14 schematically illustrates a system 100, comprising power-and-control console 70 and device 10, in accordance with a preferred embodiment of the present invention. Device 10 includes at least one, and preferably two or more spouts 12.

Preferably, power-and-control console 70 is lightweight and portable, making system 100 portable. When battery 94, which is preferably rechargeable, is used, system 100 may be used “on the go.” Preferably power-and-control console 70, device 10 and spouts 12 are arranged in an attractive carrying case, wherein power-and-control console 70 may be built into the carrying case.

In accordance with a preferred embodiment of the present invention, a plurality of spouts 12 may be provided, each having different features, adapted for a different modes of treatment and (or) different portions of the body. The different modes of treatment may include:

1. suction, macro-massage treatment, which may include one or more rollers (FIGS. 1A-1I, and 2A-2B);

2. lower-chin firming treatment (FIG. 2C);

3. breast firming treatment (FIGS. 2D-2F);

4. light treatment (FIGS. 3A and 3B), which includes:

i. warming light treatment (based on any one of infrared light, incandescent light or halogen light);

ii. UV light treatment;

iii. LLLT;

5. ultrasound treatment (FIGS. 4A-4B);

6. magnetic field treatment (FIGS. 5A-5B) which includes:

i. pulsating magnetic field treatment; and

ii. constant magnetic field treatment;

7. electrostimulation treatment (FIGS. 6A-6C);

8. cooling-diode treatment (FIG. 7);

9. warming-diode treatment (FIG. 7);

10. photoepilation (FIGS. 9A-9C);

11. mechanical epilation (FIGS. 10A-10B); and

12. impotence treatment (FIG. 12);

13. various combinations (4A, 4B, 5B and 8)

In addition, spout 12 may combine several features, such as LLLT and ultrasound (FIGS. 4A and 4B), or LLLT and pulsating magnetic field treatment (FIG. 5B). Similarly, other combinations are possible. These may be applied in tandem, in sequence, in accordance with predetermined schedules, or at different times.

For example, spout 12 may be adapted for suction massage, and may further include laser diodes tiers 56 and 58 imbedded in casing 15 (FIG. 3B), cooling diodes 47 (FIG. 7), and ultrasound transducer 60, imbedded in roller 22 (FIG. 4B). A predetermined schedule, using spout 12, may comprise,

1. a first period of 10 min.: suction, macro-massage treatment with LLLT;

2. a second period of 5 min.: ultrasound treatment; and

3. a third period of 3 min.: cooling-diode treatment.

Alternatively, spout 12 may be adapted for suction massage, and may further include infrared light 46 (FIG. 3A), cooling diodes 47 (FIG. 7) and electrostimulating electrodes 27 (FIGS. 6A-6C). A predetermined schedule, using spout 12, may comprise,

1. a first period of 8 min.: suction, macro-massage treatment with warming light treatment;

2. a second period of 8 min.: electrostimulation treatment; and

3. a third period of 3 min.: cooling-diode treatment.

It will be appreciated that many other predetermined schedules are similarly possible. Additionally, it will be appreciated that spout 12 may be used for the application of a single treatment, such as only suction, macro-massage treatment, only LLLT, or only ultrasound treatment.

Thus, in accordance with the present invention, self-application of a variety of face and body treatments is possible with device 10. The user may adhere to a predetermined schedule, or alternate between schedules, for example, a first predetermined schedule on Sundays and Tuesdays, a second predetermined schedule on Mondays and Wednesdays, and a third predetermined schedule on Fridays. Preferably, the schedules are based on clinical recommendations for the frequency and duration of different forms of treatment. Recommended predetermined schedules may be provided on diskettes, CD's, or the like, with system 100.

Preferably, device 10 further includes knob 49 (FIGS. 3A-3B) to turn off any or all features, other than vacuum, when desired. Knob 49 may be located on spout 12 or on gripping handle 14, of device 10.

It will be appreciated that spout 12 and device 14 may be integrated into a single unit, forming device 10, and a plurality of devices 10 may be provided, rather than a plurality of spouts 12.

It will be appreciated that spout 12 or device 10 may be connected to a vacuum cleaner, as described in any of FIGS. 11A-11D.

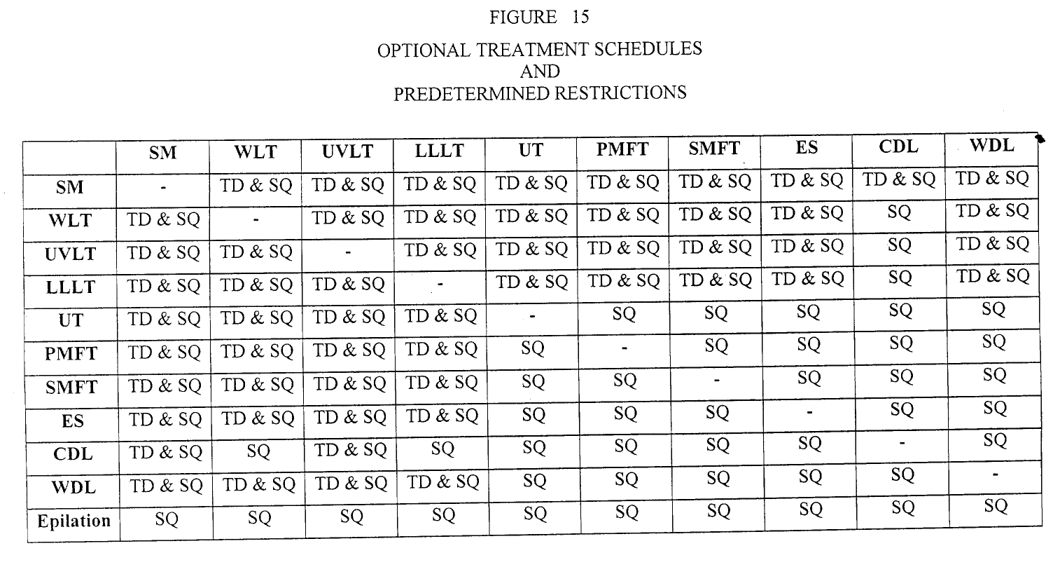

Referring further to the drawings, FIG. 15, which is arranged as a table, delineates certain predetermined restrictions regarding the different features and different modes of treatment of device 10, in accordance with the present invention. Certain modes of treatment, such as suction, macro-massage treatment and light treatment may be applied in tandem and in sequence. On the other hand, other modes may be applied only in sequence, as they interfere with each other. For example, electrostimulation is likely to interfere with the operation of a piezoelectric ultrasound transducer; thus tandem application of electrostimulation and ultrasound treatments is restricted. Similarly, cooling treatment will be ineffective, when conducted with warming light treatment, or with ultrasound treatment, both having warming effects on the tissue. In consequence, their tandem application is restricted. The key to the acronyms of FIG. 15 is as follows:

SM—suction, macro-massage treatment, with or without one or more rollers;

WLT—warming light treatment (based on any one of infrared light, incandescent light or halogen light);

UVLT—UV light treatment;

LLLT—low level laser treatment;

UT—ultrasound treatment;

PMFT—pulsating magnetic field treatment;

SMFT—constant magnetic field treatment;

ES—electrostimulation treatment;

CDL—cooling-diode treatment;

WDL—warming-diode treatment;

epilation—photoepilation or mechanical epilation;

TD & SQ—in tandem and in sequence; and

SQ—only in sequence.

It will be appreciated that a cooling system may be used with any of spouts 12, for example, as described in conjunction with FIG. 9B.

It will be appreciated that an oil or a cream, which may be for example, a baby oil, a body lotion, a hand cream, a facial cream, an aromatic oil or an aromatic cream may be used together with spout 12. However, spout 12 may also be used with no oil or cream. In accordance with another preferred embodiment of the present invention, the user may buy system 100 with a limited number of spouts 12.

Additionally, the user may upgrade his system 100 by buying additional spouts 12 at a later time.

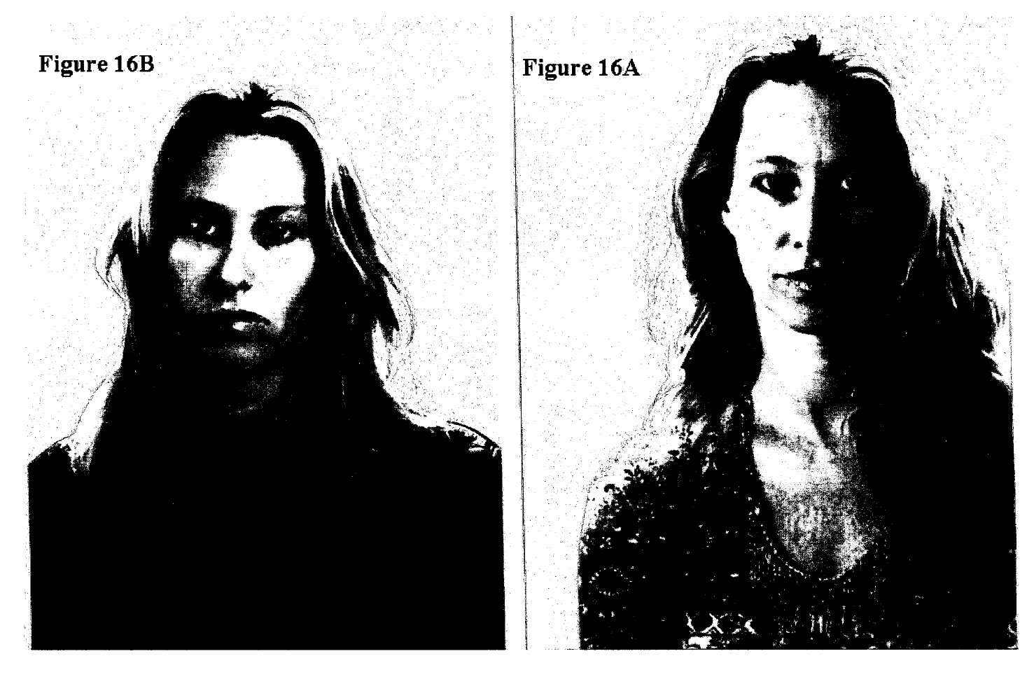

Referring further to the drawings, FIGS. 16A-16B are colored photographs of a 36-year old woman that underwent a vacuum treatment, in accordance with the present invention. FIG. 16A was taken prior to the treatment and FIG. 16B was taken after two months of treatment. The treatment schedule was as follows:

During the 1st week: 5 min., 2-3 times a week, at 0.3-0.5 of 450 mbars.

During the 2^{nd} week: 5 min., 2-3 times a week, at ˜0.6 of 450 mbars.

During the 3^{rd} week: 5 min., 2-3 times a week, at 0.9-1.0 of 450 mbars.

After the 3^{rd} week: 5 min., 2-3 times a week, at 450 mbars.

After two months: 5 min., once a week, at 450 mbars to retain the effect.

As seen when comparing FIGS. 16B and 16A, there is a marked improvement in facial tone, after two months of treatment.

It will be appreciated that the vacuum treatment schedule hereinabove is merely an example, and other treatment schedules are similarly possible.

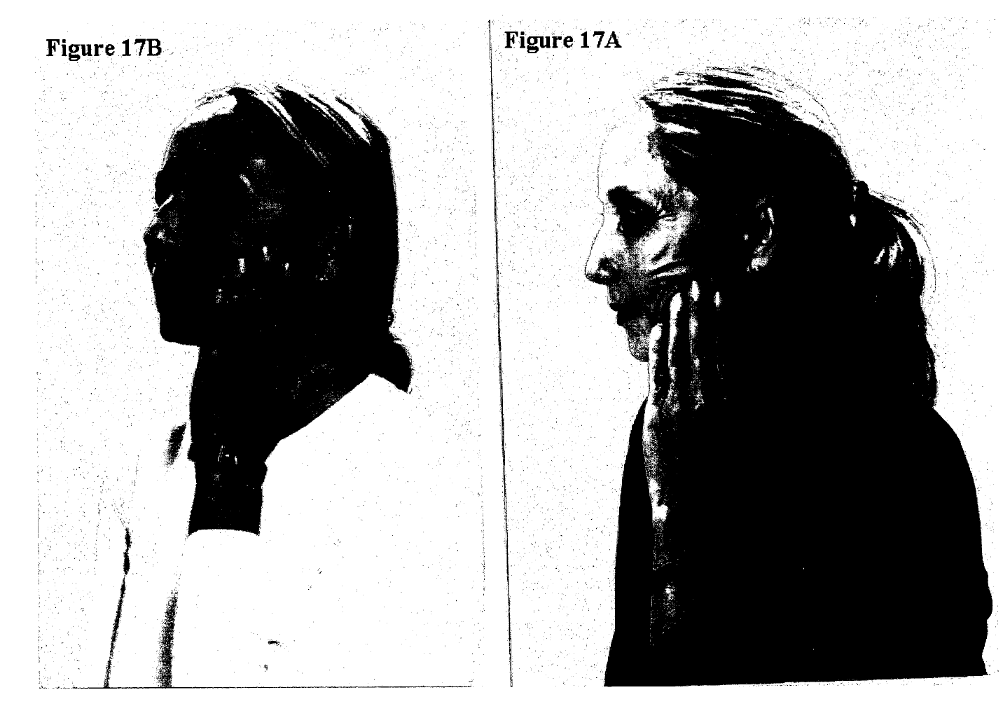

Referring further to the drawings, FIGS. 17A-17B are colored photographs of a 52-year old woman that underwent a vacuum treatment, in accordance with the present invention. FIG. 17A was taken prior to the treatment and FIG. 17B was taken after two months of treatment. The treatment schedule was as in FIGS. 16A-16B, hereinabove.

As seen when comparing FIGS. 17B and 17A, there is a marked improvement in facial tone, after two months of treatment.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims. All publications, patents and patent applications mentioned in this specification are herein incorporated in their entirety by reference into the specification to the same extent as if each individual publication, patent or patent application was specifically and individually indicated to be incorporated herein by reference. In addition, citation or identification of any reference in this application shall not be construed as an admission that such reference is available as prior art to the present invention.

What is claimed is:

1. A treatment device, comprising:
a spout, which defines an inner chamber, and which is in communication with a vacuum source, for gliding along a portion of a body and applying suction, macro-massage treatment thereto;
at least one feature, integrated with said spout, for applying at least one mode of treatment, different from macro-massage; and
a power source, in power communication with said at least one feature.

2. The device of claim 1, wherein said vacuum source is a pulsating vacuum source.

3. The device of claim 1, wherein said vacuum source is a steady vacuum source.

4. The device of claim 1, wherein said power source is a power line, and said device is detachably connected to said power line.

5. The device of claim 1, wherein said power source is a self-contained power source.

6. The device of claim 1, wherein said device is detachably connected to said vacuum source.

7. The device of claim 1, wherein said vacuum source is a vacuum cleaner.

8. The device of claim 1, wherein said vacuum source is a dedicated vacuum pump.

9. The device of claim 1, wherein said device includes a gripping handle.

10. The device of claim 9, wherein said spout is detachably connected to said gripping handle.

11. The device of claim 1, wherein said device is adapted for self-application.

12. The device of claim 1, wherein said device further includes at least one knob, for selectively activating and selectively deactivating said at least one feature, for applying said suction, macro-massage and at least one feature individually, and in tandem.

13. The device of claim 1, wherein said spout further includes at least one roller.

14. The device of claim 1, wherein said at least one feature is at least one light source, for applying light treatment to the portion of the body.

15. The device of claim 14, wherein said at least one light source is operative for photoepilation.

16. The device of claim 14, wherein said light treatment is selected from the group consisting of warming light treatment, UV light treatment, LLLT, and a combination thereof.

17. The device of claim 14, wherein said at least one light source comprises a plurality of light sources of a same type.

18. The device of claim 14, wherein said at least one light source comprises a plurality of light sources of different types.

19. The device of claim 14, wherein said at least one light source is adapted for heating the portion of the body.

20. The device of claim 14, wherein said at least one light source comprises a mercury vapor discharge lamp.

21. The device of claim 14, wherein said at least one light source comprises at least one laser light.

22. The device of claim 21, wherein said at least one laser light comprises at least two laser lights of a same wavelength.

23. The device of claim 21, wherein said at least one laser light comprises at least two laser lights of different wavelengths.

24. The device of claim 21, wherein said at least one laser light is a pulsating laser light.

25. The device of claim 14, wherein said at least one light source is operative at a wavelength range of 200-350 nm.

26. The device of claim 14, wherein said at least one light source is operative at a wavelength range of 350-600 nm.

27. The device of claim 14, wherein said at least one light source is operative at a wavelength range of 600-700 nm.

28. The device of claim 14, wherein said at least one light source is operative at a wavelength range of 700-900 nm.

29. The device of claim 14, wherein said at least one light source is operative at a wavelength range of 900-1100 nm.

30. The device of claim 14, wherein said at least one light source is operative at a wavelength range of 1100-1500 nm.

31. The device of claim 14, wherein said at least one light source is operative at a wavelength of over 1500 nm.

32. The device of claim 1, wherein said at least one feature is at least one an electromagnet, for applying a magnetic field treatment to the portion of the body.

33. The device of claim 32, wherein said magnetic field treatment is selected from the group consisting of pulsating magnetic field treatment and constant magnetic field treatment.

34. The device of claim 1, wherein said at least one feature is at least one electrode pair, for applying a electrostimulation treatment to the portion of the body.

35. The device of claim 1, wherein said at least one feature is at least one cooling diode.

36. The device of claim 35, wherein said at least one cooling diode further includes at least two cooling diodes.

37. The device of claim 35, wherein said at least one cooling diode may be flipped so as to operate as a warming diode, for providing warming-diode treatment.

38. The device of claim 35, wherein said device further includes at least one warming diode, wherein said cooling diode and said warming diode operate in cycles for cooling-warming therapy.

39. The device of claim 1, wherein said at least one feature is at least one mechanical epilation device, for applying mechanical epilation to the portion of the body.

40. The device of claim 1, wherein said spout further includes an additional feature, for applying an additional mode of treatment, selected from the group consisting of warming light treatment, UV light treatment, LLLT, ultrasound treatment, pulsating magnetic field treatment, constant magnetic field treatment, electrostimulation treatment, cooling-diode treatment, warming-diode treatment, cooling-warming-diode treatment, photoepilation, and mechanical epilation, wherein said treatments may be selectively applied in tandem and selectively applied in sequence, in accordance with predetermined restrictions.

41. The device of claim 40, wherein said treatments may be applied in accordance with a predetermined schedule.

42. A treatment system, comprising:
a treatment device, comprising:
a spout, which defines an inner chamber, and which is in communication with a vacuum source, for gliding along a portion of a body and applying suction, macro-massage treatment thereto;
at least one feature, integrated with said spout, for applying at least one mode of treatment, different from macro-massage; and
a power source, in power communication with said at least one feature; and
a power-and-control console, comprising:
an electric pump, in fluid communication with said device, for providing suction; and
a system power source, in power communication at least with said electric pump.

43. The system of claim 42, wherein said vacuum source is a pulsating vacuum source.

44. The system of claim 42, wherein said vacuum source is a steady vacuum source.

45. The system of claim 42, wherein said at least one mode of treatment is selected from the goup consisting of warming light treatment, UV light treatment, LLLT, pulsating magnetic field treatment, constant magnetic field treatment, electrostimulation treatment, cooling-diode treatment, warming-diode treatment, cooling-warming-diode treatment, photoepilation, and mechanical epilation.

46. The system of claim 42, wherein said spout includes at least two features, for applying at least two modes of treatment, different from macro-massage, wherein said treatments may be selectively applied in tandem and selectively applied in sequence, in accordance with predetermined restrictions.

47. The system of claim 46, wherein said treatments may be applied in accordance with a predetermined schedule.

48. The system of claim 42, wherein said device is detachably connected to said power-and-control console.

49. The system of claim 48, wherein said system includes a plurality of devices, of different shapes and different modes of treatment.

50. The system of claim 48, wherein said system includes a plurality of spouts, of different shapes and different modes of treatment.

51. The system of claim 42, wherein said device includes at least one roller.

52. The system of claim 42, wherein said system further includes a dedicated device to firm the lower chin.

53. The system of claim 42, wherein said system further includes a dedicated device to firm a breast.

54. The system of claim 42, wherein said system further includes a dedicated device to firm the two breasts simultaneously.

55. The system of claim 42, wherein said system further includes a dedicated device to treat impotence.

56. The system of claim 42, wherein said system is portable.

57. The system of claim 42, wherein said system power source includes a battery.

58. The system of claim 42, wherein said system includes a controller.

59. The system of claim 42, wherein said system includes a display panel.

60. The system of claim 59, wherein said display panel is interactive.

61. The system of claim 42, wherein said device is adapted for self-application.

62. A method for face and body treatment, comprising:
applying a first mode of treatment, selected from the group consisting of suction, macro-massage treatment, warming light treatment, UV light treatment, LLLT, pulsating magnetic field treatment, constant magnetic field treatment, electrostimulation treatment, cooling-diode treatment, warming-diode treatment, cooling-warming-diode treatment, photoepilation, and mechanical epilation; and
applying a second mode of treatment, different from said first mode, said second mode of treatment being selected from the group consisting of suction, macro-massage treatment, warming light treatment, UV light treatment, LLLT, pulsating magnetic field treatment, constant magnetic field treatment, electrostimulation treatment, cooling-diode treatment, warming-diode treatment, cooling-warming-diode treatment, photoepilation, and mechanical epilation,
wherein said first and second modes of treatment may be selectively applied in tandem, and selectively applied in sequence, in accordance with predetermined restrictions.

63. The method of claim 62, wherein said first and second modes of treatment are applied in accordance with a predetermined schedule.

64. The method of claim 62, wherein said modes of treatment are self-applied.
